SEQUENCE LISTING (20 sequences)

SEQ ID NO: 1 - DNA, 63 bp, synthetic construct
  source 1..63: mol_type = other DNA
ccaggcagttgagacgaacattcctaagtctgaaatttatcacccgccatagtagacgtatca

SEQ ID NO: 2 - DNA, 63 bp, synthetic construct
  source 1..63: mol_type = other DNA
cttgctacacgattcagacttaggaatgttcgacatgcgagggtccaataccgacgattacag

SEQ ID NO: 3 - DNA, 63 bp, synthetic construct
  source 1..63: mol_type = other DNA
ggtgataaaacgtgtagcaagctgtaatcgacgggaagagcatgcccatccactactatggcg

SEQ ID NO: 4 - DNA, 63 bp, synthetic construct; identical to SEQ ID NO: 1
  source 1..63: mol_type = other DNA
  misc_binding 63: bound_moiety = a cholesterol molecule

SEQ ID NO: 5 - DNA, 63 bp, synthetic construct; identical to SEQ ID NO: 2
  source 1..63: mol_type = other DNA
  misc_binding 63: bound_moiety = a cholesterol molecule

SEQ ID NO: 6 - DNA, 63 bp, synthetic construct; identical to SEQ ID NO: 3
  source 1..63: mol_type = other DNA
  misc_binding 63: bound_moiety = a cholesterol molecule

SEQ ID NO: 7 - DNA, 63 bp, synthetic construct
  source 1..63: mol_type = other DNA
cctcgcatgactcaactgcctggtgatacgaggatgggcatgctcttcccgacggtattggac

SEQ ID NO: 8 - DNA, 88 bp, synthetic construct
  misc_feature 87..88: note = RNA; note = G is 2'-O-methoxy-RNA
  source 1..88: mol_type = other DNA
  misc_feature 1..86: note = DNA
cctcgcatgactcaactgcctggtgatacgaggatgggcatgctcttcccgacggtattggactttttgtccaccattagcacgcggg

SEQ ID NO: 9 - DNA, 88 bp, synthetic construct
  misc_feature 87..88: note = RNA; note = g is 2'-O-methoxy-RNA
  source 1..88: mol_type = other DNA
  misc_feature 1..86: note = DNA
cctcgcatgactcaactgcctggtgatacgaggatgggcatgctcttcccgacggtattggactttttggtggtgttggtggtggtgg

SEQ ID NO: 10 - DNA, 20 bp, synthetic construct
  source 1..20: mol_type = other DNA
gtccaccattagcacgcggg

SEQ ID NO: 11 - DNA, 20 bp, synthetic construct
  source 1..20: mol_type = other DNA
  misc_binding 20: bound_moiety = a cholesterol molecule
gtccaccattagcacgcggg

SEQ ID NO: 12 - DNA, 20 bp, synthetic construct
  source 1..20: mol_type = other DNA
  misc_binding 1: bound_moiety = GalNAc3
gtccaccattagcacgcggg

SEQ ID NO: 13 - DNA, 22 bp, synthetic construct
  source 1..22: mol_type = other DNA
tacctgaacccgtgttgctctc

SEQ ID NO: 14 - DNA, 22 bp, synthetic construct
  source 1..22: mol_type = other DNA
gttgctgaggtatcgccaggaa

SEQ ID NO: 15 - DNA, 20 bp, synthetic construct
  source 1..20: mol_type = other DNA
agagctacgagctgcctgac

SEQ ID NO: 16 - DNA, 20 bp, synthetic construct
  source 1..20: mol_type = other DNA
agcactgtgttggcgtacag

SEQ ID NO: 17 - DNA, 20 bp, synthetic construct
  source 1..20: mol_type = other DNA
cactcccgtggcttctagtg

SEQ ID NO: 18 - DNA, 20 bp, synthetic construct
  source 1..20: mol_type = other DNA
gcgggtgacctctttagcat

SEQ ID NO: 19 - DNA, 20 bp, synthetic construct
  source 1..20: mol_type = other DNA
gccatgtacgtagccatcca

SEQ ID NO: 20 - DNA, 20 bp, synthetic construct
  source 1..20: mol_type = other DNA
atggcgtgagggagagcata

(19) United States
(12) Patent Application Publication
(10) Pub. No.: US 2024/0115515 A1
(43) Pub. Date: Apr. 11, 2024

(54) LIVER-SPECIFIC DRUG DELIVERY CARRIER COMPRISING NUCLEIC ACID NANOPARTICLE AND CHOLESTEROL AND USE THEREOF

(71) Applicant: KOREA INSTITUTE OF SCIENCE AND TECHNOLOGY, Seoul (KR)

(72) Inventors: Dae-Ro AHN, Seoul (KR); Kyoung-Ran KIM, Seoul (KR); Junghyun KIM, Seoul (KR); Ji Eun LEE, Seoul (KR); Ji hyun BACK, Seoul (KR)

(73) Assignee: KOREA INSTITUTE OF SCIENCE AND TECHNOLOGY, Seoul (KR)

(21) Appl. No.: 18/166,782

(22) Filed: Feb. 9, 2023

(30) Foreign Application Priority Data
Feb. 9, 2022 (KR) 10-2022-0017065

(51) Int. Cl. A61K 9/51; A61K 47/54; A61P 1/16; C12N 15/113

(52) U.S. Cl. CPC A61K 9/5169; A61K 47/549; A61P 1/16; C12N 15/113

(57) ABSTRACT

The present invention relates to a liver-specific drug delivery carrier comprising a nucleic acid nanoparticle and cholesterol; a liver-specific complex; a pharmaceutical composition for prevention or treatment of liver disease using the same; and a method for preventing or treating liver disease.

### CROSS REFERENCE TO RELATED APPLICATIONS

This application claims the benefit under 35 USC 119(a) of Korean Patent Application No. 10-2022-0017065 filed on Feb. 9, 2022, in the Korean Intellectual Property Office, the entire disclosure of which is incorporated herein by reference for all purposes.

### REFERENCE TO AN ELECTRONIC SEQUENCE LISTING

The contents of the electronic sequence listing (“NewApp__0210920010__SegListing.xml”; Size is 23 kilobytes and it was created on Feb. 8, 2023) is herein incorporated by reference in its entirety.

### TECHNICAL FIELD

The present invention relates to a liver-specific drug delivery carrier comprising a nucleic acid nanoparticle and cholesterol; a liver-specific complex; a pharmaceutical composition for prevention or treatment of liver disease using the same; and a method for preventing or treating liver disease.

### BACKGROUND ART

Recently, as mass production by mass synthesis or genetic engineering has become possible, various nucleic acids, proteins, peptides, enzymes, and the like are used as drugs exhibiting high therapeutic effects. However, drugs may be easily hydrolyzed or destroyed by enzymes in a short period of time after being administered into the body, and the uptake rate thereof may be extremely low. In addition, when the drugs are repeatedly administered, an immune response is frequently induced, and a life-threatening hypersensitivity reaction due to the neutralization of physiological activity by the produced antibody may be caused, and also, clearance by the reticuloendothelial system may increase. Accordingly, various studies are being conducted to improve the limitations in applying drugs required to target hepatocytes, but existing or new limitations such as causing of side effects have not yet been overcome.

For example, Hashida et al. have prepared a polymeric prodrug by modifying biodegradable polymers such as poly-L-glutamic acid (PLGA) and galactosylated poly-L-lysine to poorly soluble and unstable prostaglandin, and then increased liver targeting of the drug (J. of Contr. Release, 62, 253-262, 1999). However, there are no accurate reports on the stability and immunogenicity of the drug when the used polymer is degraded.

Therefore, there is a need for a new method in which the uptake of a drug, which is required to target hepatocytes, into the body increases, the drug targeting hepatocytes in the body reach the target site at a high rate, and thus the efficacy of the drug increases, and the side effects of the drug decrease.

In particular, the market is expected to expand along with the recent increase among drugs in FDA-approved oligo drugs. The market size of liver disease, more specifically liver fibrosis disease, which is an incurable disease, is expanding, and the global market size of oligonucleotide therapeutics is expected to continue to increase. However, in this technical field as well, the technology used for hepatocyte/tissue-selective delivery of oligonucleotide therapeutics which is under clinical trials or approved is extremely limited, and thus requires development.

### DISCLOSURE

### Technical Problem

The present inventors have revealed that a drug delivery carrier comprising a nucleic acid nanoparticle and cholesterol exhibits liver specificity, and confirmed that liver tissue/hepatocyte-specific drug delivery remarkably increases when the drug delivery carrier is used, thereby completing the present invention.

### Technical Solution

An object of the present invention is to provide a liver-specific drug delivery carrier comprising a nucleic acid nanoparticle and cholesterol.

Another object of the present invention is to provide a liver-specific complex comprising a nucleic acid nanoparticle and cholesterol.

Still another object of the present invention is to provide a pharmaceutical composition for prevention or treatment of liver disease comprising the drug delivery carrier and a pharmaceutically active ingredient.

Still another object of the present invention is to provide a method for preventing or treating liver disease using the pharmaceutical composition.

### Advantageous Effects

When the present invention is used, selective delivery to hepatocytes/liver tissue is possible, and thus the effect of preventing or treating liver diseases is remarkably excellent and side effects decrease, and as a result, the present invention can be widely used particularly as a base technology for drug development.

### BRIEF DESCRIPTION OF DRAWINGS

FIG. 1A is a diagram illustrating the method of action when a complex of a nucleic acid nanoparticle and cholesterol is administered to a subject and a schematic diagram of a protein corona produced at this time;

FIG. 1B is a schematic diagram illustrating the structures of a nucleic acid nanoparticle (Td) and a trivalent complex (Chol₃-Td) of a nucleic acid nanoparticle and cholesterol;

FIG. 2 is a diagram illustrating electrophoresis results of Td, Chol₁-Td, Chol₂-Td, and Chol₃-Td;

FIG. 3 is a diagram illustrating the results of dynamic light scattering (DLS) analysis of Td and Chol₃-Td depending on lipoprotein (HDL or LDL) binding;

FIG. 4 is a diagram illustrating the results of atomic force microscopy (AFM) analysis of Td and Chol₃-Td depending on lipoprotein (HDL or LDL) binding;

FIG. 5 is a diagram illustrating the size analysis results of Td, Chol₃-Td, and lipoprotein complexes of these measured from representative particles (50 to 100) in atomic force microscope images;

FIG. 6 is a diagram illustrating the results of analyzing the stability of Td and Chol₃-Td with respect to nucleases depending on lipoprotein (HDL or LDL) binding (C: control);

FIG. 7 is a schematic diagram illustrating a pull-down assay process;

FIG. 8 is a diagram illustrating the SDS-PAGE analysis results of DNA nanoparticles and pulled-down mouse serum proteins;

FIG. 9 is a diagram illustrating the results of Western blotting analysis confirming that serum proteins are absorbed by Chol₃-Td;

FIGS. 10A and 10B are diagrams illustrating the results of analyzing the dissociation constants of lipoproteins (HDL, LDL) and major serum proteins (albumin. IgG) in Chol₃-Td and Td based on SDA PAGE;

FIG. 11 is a diagram illustrating a heat map acquired by hierarchically clustering proteins of pulled-down samples;

FIGS. 12 and 13 are diagrams illustrating the results of confirming the behavior of Cy5-labeled Td and Cy5-labeled Chol₃-Td in vivo over time after intraperitoneal injection thereof and the ex vivo results confirming the distribution of Cy5-labeled Td and Cy5-labeled Chol₃-Td in major organs after harvesting;

FIG. 14 is a diagram illustrating the results of quantifying the distribution of Cy5-labeled Td and Cy5-labeled Chol₃-Td in organs;

FIG. 15 is a diagram illustrating the results of confocal fluorescence microscopy analysis showing the distribution of Td and Chol₃-Td depending on the liver cell type;

FIG. 16 is a diagram illustrating the results of quantifying the distribution of Td and Chol₃-Td depending on the liver cell type (HC: hepatocyte; MP: microphage; and EC: endothelial cell);

FIG. 17 is a diagram illustrating the results of quantifying the uptake of Td, Chol₁-Td, Chol₂-Td, and Chol₃-Td into cells depending on the liver cell type and serum protein type (HDL, LDL, albumin, IgG);

FIG. 18 is a diagram illustrating the results of confocal fluorescence microscopy analysis showing uptake of Chol₃-Td, Chol₃-Td/LDL, and Chol₃-Td/HDL into hepatocytes depending on the treatment with anti-SR-B1 antibody and anti-LDLR antibody;

FIG. 19 is a diagram illustrating the results of quantifying uptake of Chol₃-Td, Chol₃-Td/LDL, and Chol₃-Td/HDL into hepatocytes depending on the treatment with anti-SR-B1 antibody and anti-LDLR antibody;

FIG. 20 is a diagram illustrating the results of cytotoxicity evaluation of Chol₃-Td and Td;

FIG. 21 is a schematic diagram illustrating the structure of a trivalent complex (ASO@Chol₃-Td) of an antisense oligonucleotide (ASO), a nucleic acid nanoparticle, and cholesterol;

FIG. 22 is a diagram illustrating the results of confirming TGF-β1 mRNA (RT-PCR) and protein expression levels (Western blotting) in hepatocytes;

FIG. 23 is a diagram illustrating a positive correlation between gene silencing and cellular uptake efficiency;

FIG. 24 is a diagram illustrating the results of ex vivo imaging to confirm the liver-specific organ distribution of ASO@Chol₃-Td;

FIG. 25 is diagrams illustrating the results of confocal fluorescence microscopy analysis showing the distribution of ASO@Chol₃-Td in a mouse with liver fibrosis depending on the liver cell type and the relative localization rate of ASO@Chol₃-Td;

FIG. 26 is a schematic diagram illustrating a method for constructing a liver fibrosis mouse model and performing treatment with ASO;

FIG. 27A is a diagram illustrating the results of confirming TGF-β1 mRNA (RT-PCR) and protein expression levels (Western blotting) to confirm the therapeutic effect of ASO in vivo in a mouse with liver fibrosis and its ability to regulate target gene expression (62 μg/kg per injection) (GAPDH is an internal control for determination of TGF-β1 mRNA and protein levels);

FIG. 27B is a graph acquired by quantifying the Western blotting image of FIG. 27A;

FIG. 28 is a diagram illustrating immunofluorescence analysis results of TGF-β1 expression (red) in liver sections; results of myofibroblasts (measured by α-smooth muscle actin: α-SMA expression, green); (blue; DAPI, nuclei); and the mean fluorescence intensities (MFI) acquired by quantitatively analyzing the red and green wavelengths (mean±SD, n=4);

FIG. 29A is a diagram illustrating images acquired through tissue staining (Sirius red);

FIG. 29B is a diagram illustrating the results of quantifying collagen-rich regions in stained tissue images;

FIG. 29C is a diagram illustrating Western blotting images for additional confirmation of collagen level;

FIG. 29D is a graph acquired by quantifying the Western blotting images of FIG. 29C;

FIG. 30 is a diagram illustrating the serological analysis results (AST, ALT) of serum isolated from mouse blood samples;

FIG. 31 is a diagram illustrating the results of comparing the final weights of livers harvested after the end of treatment;

FIG. 32 is a diagram illustrating the chemical structure of GalNAc₃ and a schematic diagram of GalNAc₃-ASO;

FIG. 33 is a diagram illustrating the results of electron spray ionization (ESI) mass spectrometry of GalNAc₃-ASO;

FIG. 34 is a diagram illustrating the results of analyzing TGF-β1 mRNA and protein levels when hepatocytes are treated with GalNAc₃-ASO and ASO@Chol₃-Td, respectively;

FIG. 35 is a diagram illustrating the results of analyzing TGF-β1 mRNA and protein levels in vivo when treated with GalNAc₃-ASO and ASO@Chol₃-Td, respectively;

FIG. 36 is a diagram illustrating the results of observation of in vivo behavior of Chol₃-Td and ASO@Chol₃-Td over time after tail vein injection thereof and the results of ex vivo imaging showing the distribution of Chol₃-Td and ASO@Chol₃-Td in major organs after organ harvesting in healthy mice and liver fibrosis model mice;

FIG. 37 is a diagram illustrating the results of analyzing TGF-β1 mRNA and protein levels in vivo when ASO@Chol₃-Td is administered by tail vein injection compared to those when ASO@Chol₃-Td is injected intraperitoneally; and

FIG. 38 is a diagram illustrating ex vivo imaging results to analyze the extent to which Chol₃-Td and Chol₃-Td complexed with lipoproteins reach the liver when Chol₃-Td and Chol₃-Td complexed with lipoproteins are orally administered, respectively.

### DETAILED DESCRIPTION OF THE INVENTION

Hereinafter, the contents of the present invention will be described in detail as follows. Meanwhile, the description and embodiments of an aspect disclosed in the present invention can also be applied to the description and embodiments of another aspect with respect to common matters. Moreover, all combinations of the various elements disclosed in the present invention fall within the scope of the present invention. In addition, documents described in the present invention may be incorporated herein by reference. Additionally, it cannot be seen that the scope of the present invention is limited by the specific descriptions described below.

An aspect of the present invention provides a liver-specific drug delivery carrier comprising a nucleic acid nanoparticle and cholesterol.

As used herein, the term “nanoparticle” broadly refers to a particle having a diameter of several to several hundred nanometers. The manufacturing method thereof may be largely divided into three methods: a top-down approach, which is a physical method, and a bottom-up approach and a self-assembly method, which are based on chemical synthesis. Among these, the self-assembly method is currently the basis for assembly in biomolecular nanotechnology, and is a kind of bottom-up approach. The components of self-assembling particles may spontaneously aggregate to form nanoparticles by physical, chemical, and structural properties. At this time, the particle size may be determined by adjusting the molar ratio among the reactants. In addition, the physical properties of formed nanoparticles may be improved by modifying the surface, and then the formed nanoparticles may be applied to various fields.

For the purpose of the present invention, the nanoparticle is a nucleic acid nanoparticle containing a nucleic acid, and may be a nucleic acid nanoparticle composed of a nucleic acid, but is not limited thereto.

In an embodiment, the nucleic acid nanoparticle may have a three-dimensional structure, and may be composed of a tetrahedral structure, but is not limited thereto.

In an embodiment, the nucleic acid nanoparticle may be a three-dimensional self-assembling nucleic acid nanoparticle, but is not limited thereto.

In an embodiment, the nucleic acid nanoparticle may be composed of any one or more oligonucleotides selected from the group consisting of nucleotide sequences of SEQ ID NOs: 1 to 8, but does not exclude meaningless sequence additions to before and after the nucleotide sequence of SEQ ID NOs: 1 to 8, naturally occurring mutations, or silent mutations thereof, and it is apparent to those skilled in the art that an oligonucleotide composed of a nucleotide sequence in which some sequences are deleted, modified, substituted, or added also corresponds to the oligonucleotide of the present invention as long as it exhibits activity the same as or corresponding to that of an oligonucleotide including the nucleotide sequence of SEQ ID NOs: 1 to 8. As a specific example, the oligonucleotide of the present invention may be composed of the nucleotide sequence of SEQ ID NOs: 1 to 8, or a nucleotide sequence having 80%, 85%, 90%, 95%, 96%, 97%, 98%, or 99% or more homology or identity to the nucleotide sequence of SEQ ID NOs: 1 to 8. In addition, it is apparent that an oligonucleotide composed of a nucleotide sequence in which some sequences are deleted, modified, substituted, or added is also included within the scope of the oligonucleotide of the present application as long as it is an oligonucleotide having such homology or identity and exhibiting an efficacy corresponding to that of the oligonucleotide described above.

As used herein, the term “oligonucleotide” refers to a synthesized short-stranded DNA or RNA molecule.

In an embodiment, the oligonucleotide contained in the nanoparticle may be single-stranded, but is not limited thereto.

The oligonucleotide contained in the nanoparticle may include a form in which any one or more of the nucleotides constituting the oligonucleotide are chemically or physically modified. The chemical or physical modification may include any one or more modifications selected from the group consisting of, for example, substitution with optical isomers, sugar-modified nucleotides, base-modified nucleotides, phosphorothioate nucleic acids, phosphorodithioate nucleic acids, phosphoramidate nucleic acids, amide-linked nucleic acids, MMI-linked nucleic acids, alpha-nucleic acids, and methylphosphonate nucleic acids, but is not limited thereto, and may include modifications known in the art without limitation.

The substitution with optical isomers may be, for example, substitution with any one or more of D-DNA or L-DNA, but is not limited thereto.

The sugar-modified nucleotides may be, for example, 2′-fluoro-RNA, 2′-O-methoxy-RNA, 2′-amino RNA, 2′-O-alkyl nucleic acid, 2′-O-allyl nucleic acid, 2′-O-alkynyl nucleic acid, hexose nucleic acid, pyranosyl RNA, and anhydrohexitol nucleic acid, and may be specifically any one or more of 2′-fluoro-RNA or 2′-O-methoxy-RNA, but is not limited thereto.

For the purpose of the present invention, all of the nucleotides constituting the oligonucleotide contained in the nanoparticle may be composed of any one selected from the group consisting of D-DNA, L-DNA, 2′-fluoro-RNA, and 2′-O-methoxy-RNA, but is not limited thereto.

The nucleic acid nanoparticle may be formed by self-assembly of a plurality of single-stranded nucleic acids, for example, 4 to 100, 4 to 50, or 4 to 20 stranded nucleic acids by hybridization principle. In an embodiment, the nucleic acid nanoparticle may be composed of four oligonucleotides, but is not limited thereto.

In an embodiment, the four oligonucleotides constituting the nucleic acid nanoparticle may include four kinds of oligonucleotides selected from the group consisting of the nucleotide sequences of SEQ ID NOs: 1 to 8. For example, the four kinds of oligonucleotides may include oligonucleotides composed of the nucleotide sequences of SEQ ID NOs: 1 to 4, but are not limited thereto, and the four kinds of oligonucleotides may include oligonucleotides composed of the nucleotide sequences of SEQ ID NOs: 1 to 3 and 7, but are not limited thereto. In addition, SEQ ID NOs: 4 to 6 may include cholesterol molecules.

The oligonucleotide composed of nucleotide sequences of SEQ ID NOs: 1 to 8 may include the above-described chemical or physical modifications, and may be specifically composed of any one of 2′-fluoro-RNA or 2′-O-methoxy-RNA, but is not limited thereto.

For the purpose of the present invention, the oligonucleotide of SEQ ID NOs: 1 to 8 may include one in which the entire nucleotide sequence constituting the oligonucleotide is composed of any one of 2′-fluoro-RNA or 2′-O-methoxy-RNA, but is not limited thereto.

In an embodiment, the nucleic acid nanoparticle may include a double-stranded nucleic acid containing a hybridization region in which the above-described oligonucleotide and an oligonucleotide hybridizing thereto are hybridized, and the double-stranded nucleic acid may constitute one side of the nucleic acid nanoparticle structure, but the nucleic acid nanoparticle is not limited thereto.

However, the present invention is significant in that it has developed a complex containing both a nucleic acid nanoparticle and cholesterol for the first time and revealed that the complex has liver tissue and/or hepatocyte specificity and thus has drug delivery ability, and thus the nucleic acid nanoparticle may include any nanoparticles containing nucleic acids, regardless of their manufacturing method (for example, physical method, chemical method, self-assembly) and their sequence.

The drug delivery carrier of the present invention contains cholesterol.

The cholesterol may be linked to the nucleic acid nanoparticle. The cholesterol may be linked to the nucleic acid nanoparticle directly or through a linker, or may further contain other protein (for example, cell-penetrating protein) moieties, but is not limited thereto. As the method for linking cholesterol to the nucleic acid nanoparticle of the present invention, methods performed in the art may be used without limitation as long as the activity of the nucleic acid nanoparticle is not changed. The linker may be a peptidic linker or a non-peptidic linker composed of 1 to 10 amino acids, but is not limited thereto. The drug delivery carrier of the present invention may include binding of a substance capable of increasing its half-life or introduction of mutations to prevent its degradation in the body.

In an embodiment, the cholesterol may be linked to the 3′ end of the oligonucleotide constituting the nucleic acid nanoparticle, but is not limited thereto.

In an embodiment, the drug delivery carrier may contain 1 to 4 cholesterol molecules, but the number of cholesterol molecules is not limited.

The drug delivery carrier of the present invention may form a protein layer by being bound to a serum protein. Specifically, the drug delivery carrier of the present invention may form a protein corona by being bound to a serum protein in the body, and the serum protein may be a lipoprotein or a protein related thereto, but is not limited thereto.

As used herein, the term “protein corona” means a protein layer formed on the surface when nucleic acid nanoparticles are administered into the body. The protein corona may be one of the main factors determining the lifespan of nanoparticles in vivo, and may facilitate a targeted drug delivery system.

In an embodiment, the complex and drug delivery carrier containing a nucleic acid nanoparticle and cholesterol of the present invention may form a protein corona in the body of a subject.

The protein of the protein corona may include any protein without limitation as long as it is a serum protein, but may include more specifically albumin, immunoglobulin, and lipoprotein, still more specifically, high-density lipoprotein (HDL) and/or low-density lipoprotein (LDL).

In Examples of the present invention, it was confirmed that the drug delivery carrier of the present invention forms a protein corona in the body as the drug delivery carrier contains a nucleic acid nanoparticle and cholesterol. In this case, cholesterol linked to the nucleic acid nanoparticle binds to a serum protein (particularly, lipoprotein) to form a protein corona, and thus the liver tissue and/or hepatocyte uptake and specificity may increase.

In addition, even if the size of the protein corona is not significantly different from the size of the liver sinusoid endothelial fenestrae (100 nm to 140 nm), the nucleic acid nanoparticle and lipoprotein structures may be modified, and thus the protein corona may easily pass through the liver sinusoid endothelial fenestrae.

The drug delivery carrier may further contain a pharmaceutically active ingredient.

The pharmaceutically active ingredient may be delivered specifically to liver tissue and/or hepatocytes by the drug delivery carrier of the present invention, and may be linked to the nucleic acid nanoparticle, but is not limited thereto. In an embodiment, the pharmaceutically active ingredient may be delivered after being bound to the nucleic acid backbone of the nucleic acid nanoparticle or captured inside the nucleic acid nanoparticle, but is not limited thereto.

The pharmaceutically active ingredient may be any one or more of a drug or a nucleic acid, and may have a preventive or therapeutic use for a disease, and for example, the drug may be contrast mediums, hormones, anti-hormonal agents, vitamins, calcium preparations, inorganic preparations, saccharide preparations, organic acid preparations, protein amino acid preparations, antidotes, enzyme preparations, metabolic preparations, tissue regenerating agents, chlorophyll preparations, pigment preparations, radiopharmaceuticals, tissue cell diagnosis agents, tissue cell therapeutic agents, antibiotic agents, antiviral agents, combined antibiotic agents, chemotherapeutic agents, vaccines, toxins, toxoids, antitoxins, leptospira serum, blood products, biological products, analgesics, immunogenic molecules, antihistamines, allergy medicines, non-specific immunogen preparations, anesthetics, stimulants, psychotropic agents, and peptides, but is not limited thereto. The nucleic acid may be oligonucleotide-based one, but is not limited thereto, and the nucleic acid may be any one or more selected from aptamer, siRNA, miRNA, mRNA, CIRSPR/Cas, antisense nucleic acid, and antisense oligonucleotide (ASO), but is not limited thereto.

In an embodiment, the pharmaceutically active ingredient may inhibit a gene that upregulates an apoptosis pathway, and for example, the gene that upregulates an apoptosis pathway may be any one or more selected from the group consisting of p53. Fas, TRAIL (tumor necrosis factor-related apoptosis-inducing ligand), tumor necrosis factor (TNF), and their receptors, bcl-2 and caspase, specifically p53, but is not limited thereto.

As used herein, the term “antisense oligonucleotide (ASO)” refers to a single-stranded DNA or RNA complementary to a specific gene sequence, and is utilized to decrease the level of protein synthesis by inhibiting the processing and translation of mRNA. When ASO acts normally, the DNA/RNA double-stranded portion between mRNA and ASO is degraded by RNase H enzyme, so that the processing and translation of mRNA may be inhibited. Gene expression may be decreased using a molecular tool called morpholinos that causes RNA splicing proteins to be blocked. The antisense oligonucleotide may be used in combination with antisense.

In the present invention, the specific gene sequence may be a gene sequence encoding transforming growth factor beta (TGF-β) including TGF-β1, 2, and 3 subfamilies, specifically a gene sequence encoding any one or more selected from the group consisting of TGF-β1, TGF-β2 and TGF-β3, more specifically a gene sequence encoding TGF-β1, but is not limited thereto.

In the present invention, the antisense for TGF-β may be specifically antisense for any one or more selected from the group consisting of TGF-β1, TGF-β2 and TGF-β3, more specifically antisense for TGF-β1, but is not limited thereto.

In an embodiment, the antisense for TGF-β may include or consist of the sequence of SEQ ID NO: 10, but is not limited thereto.

Another aspect of the present invention provides a liver-specific complex comprising a nucleic acid nanoparticle and cholesterol.

The nucleic acid nanoparticle and cholesterol are as described in other embodiments.

As the drug delivery carrier of the present invention, the liver-specific complex of the present invention may further contain a pharmaceutically active ingredient. The liver specificity of the present invention is to have a high preference or selectivity for liver tissue and/or hepatocytes compared to other organs and tissues, and may be caused by the formation of a protein corona through binding of cholesterol linked to the nucleic acid nanoparticle to a protein in the blood.

Still another aspect of the present invention provides a pharmaceutical composition for prevention or treatment of liver disease comprising a liver-specific drug delivery carrier containing a nucleic acid nanoparticle and cholesterol and a pharmaceutically active ingredient.

The nanoparticle, liver-specific drug delivery carrier, and pharmaceutically active ingredient are as described in other embodiments.

As used herein, the term “liver disease” generally encompasses diseases that occur in the liver, including liver damage.

In an embodiment, the liver disease of the present invention may be any one or more selected from the group consisting of liver damage, liver fibrosis, liver inflammation, liver cirrhosis, hepatitis, liver decompensation, steatosis, and liver cancer, but is not limited thereto.

In Examples of the present invention, liver fibrosis improving and treating effects were confirmed, but it is apparent that various liver diseases can be treated by modifying the kind of pharmaceutically active ingredient and the disease, protein, or gene targeted by the ingredient as long as the pharmaceutical composition contains a pharmaceutically active ingredient together with a nucleic acid nanoparticle and cholesterol and forms a protein corona as a result, and the liver disease is not limited to a specific liver disease.

As used herein, the term “prevention” refers to any action that inhibits or delays the development or progression of liver disease by administering the composition of the present invention.

As used herein, the term “treatment” refers to clinically intervening to alter the natural process of a subject or cell to be treated, and may be performed while the clinical pathology is progressing or to prevent the progression. Desired treatment effects include preventing occurrence or recurrence of the disease, alleviating symptoms, reducing any direct or indirect pathological consequences of the disease, decreasing the rate of disease progression, alleviating or palliating the disease state, remission, or improving the prognosis. Specifically, treatment includes any action that improves liver disease by administering the composition of the present invention.

For prophylactic use, the pharmaceutical composition of the present invention is administered to a subject suspected of having or at risk of developing a disease, disorder, or condition described herein. In other words, the pharmaceutical composition may be administered to a subject at risk of developing liver disease. For therapeutic use, the pharmaceutical composition of the present invention is administered to a subject, such as a patient already suffering from a disorder described herein, in an amount sufficient to treat or at least partially arrest the symptoms of a disease, disorder, or condition described herein. The amount effective for such use depends on the severity and course of the disease, disorder or condition, previous treatments, the subject's health status and responsiveness to drugs, and the judgment of the physician or veterinarian.

The pharmaceutical composition may contain a pharmaceutically acceptable carrier. The “pharmaceutically acceptable carrier” may refer to a carrier, excipient, or diluent that does not inhibit the biological activity and properties of the compound to be injected as well as does not irritate the organism, and may be specifically a non-naturally occurring carrier. The kind of carrier that can be used in the present invention is not particularly limited, and any carrier that is commonly used in the art and pharmaceutically acceptable may be used. Non-limiting examples of the carrier include saline, sterile water, Ringer's solution, buffered saline, albumin injection solution, dextrose solution, maltodextrin solution, glycerol, and ethanol. These may be used singly or in combination of two or more kinds thereof.

The composition containing a pharmaceutically acceptable carrier may be in various oral or parenteral formulations. In the case of being formulated into preparations, the preparations are prepared using diluents or excipients such as fillers, extenders, binders, wetting agents, disintegrants, and surfactants commonly used.

Specifically, solid preparations for oral administration include tablets, pills, powders, granules, and capsules, and such solid preparations may be prepared by mixing the compound with at least one or more excipients, for example, starch, calcium carbonate, sucrose, lactose, and gelatin. In addition to simple excipients, lubricants such as magnesium stearate and talc may also be used. Liquid preparations for oral administration include suspensions, solutions for internal use, emulsions, and syrups, and may contain various excipients, for example, wetting agents, sweeteners, aromatics, and preservatives in addition to water and liquid paraffin, which are commonly used simple diluents. Preparations for parenteral administration include sterilized aqueous solutions, non-aqueous solutions, suspensions, emulsions, freeze-dried preparations, and suppositories. Propylene glycol, polyethylene glycol, vegetable oils such as olive oil, and injectable esters such as ethyl oleate may be used as non-aqueous solvents and suspending agents. As a base for suppositories, Witepsol, Macrogol, Tween 61, cacao butter, laurin, glycerogelatin, and the like may be used.

The content of the active ingredient contained in the composition of the present invention may be 0.0001 wt % to 10 wt %, preferably 0.001 wt % to 1 wt % with respect to the total weight of the composition, but is not particularly limited thereto.

The pharmaceutical composition may be in any one formulation selected from the group consisting of tablets, pills, powders, granules, capsules, suspensions, solutions for internal use, emulsions, syrups, sterilized aqueous solutions, non-aqueous solutions, suspensions, emulsions, freeze-dried preparations, and suppositories, and may be in various oral or parenteral formulations. In the case of being formulated into preparations, the preparations are prepared using diluents or excipients such as fillers, extenders, binders, wetting agents, disintegrants, and surfactants commonly used. Solid preparations for oral administration include tablets, pills, powders, granules, and capsules, and such solid preparations are prepared by mixing one or more compounds with at least one or more excipients, for example, starch, calcium carbonate, sucrose or lactose, and gelatin. In addition to simple excipients, lubricants such as magnesium stearate and talc are also used. Liquid preparations for oral administration include suspensions, solutions for internal use, emulsions, and syrups, and may contain various excipients, for example, wetting agents, sweeteners, aromatics, and preservatives in addition to water and liquid paraffin, which are commonly used simple diluents. Preparations for parenteral administration include sterilized aqueous solutions, non-aqueous solutions, suspensions, emulsions, freeze-dried preparations, and suppositories. Propylene glycol, polyethylene glycol, vegetable oils such as olive oil, and injectable esters such as ethyl oleate may be used as non-aqueous solvents and suspending agents. As a base for suppositories, Witepsol, Macrogol, Tween 61, cacao butter, laurin, glycerogelatin, and the like may be used.

The composition of the present invention may be administered to a subject in a pharmaceutically effective amount.

As used herein, the term “pharmaceutically effective amount” means an amount sufficient to treat a disease with a reasonable benefit/risk ratio applicable to medical treatment, and the effective dosage level may be determined depending on factors including the kind, severity, age, sex of the subject, the type of the disease, the activity of the drug, the sensitivity to the drug, the time of administration, the route of administration and the excretion rate, the duration of treatment, and the drugs used concurrently, and other factors well known in the medical arts. The composition of the present invention may be administered as an individual therapeutic agent or in combination with other therapeutic agents, and may be administered sequentially or simultaneously with conventional therapeutic agents. The composition of the present invention may be administered singly or in multiple doses. It is important to administer the composition in a minimum amount that can afford the maximum effect without causing side effects in consideration of all the above factors, and the amount can be easily determined by those skilled in the art. The preferred dosage of the composition of the present invention varies depending on the condition and body weight of the patient, the severity of the disease, the type of drug, and the route and duration of administration, and the administration may be performed once a day or several times a day in a divided manner. The composition can be applied to any one as long as it is a subject of which the liver disease is intended to be prevented or treated without particular limitation. The method of administration includes any method without limitation as long as it is a conventional method in the art. For example, the administration may be by oral, rectal or intravenous, intramuscular, subcutaneous, intraperitoneal, intrauterine intrathecal, or intracerebroventricular injection.

The pharmaceutical composition of the present invention can be administered daily or intermittently, and can be administered once or 2 to 3 times in a divided manner in terms of the number of administrations per day.

The pharmaceutical composition of the present invention may be administered to a subject who has developed liver disease or has a high possibility of developing liver disease to prevent occurrence of liver disease or alleviate the severity of liver disease.

Still another aspect of the present invention provides a method for preventing or treating liver disease, the method comprising administering the pharmaceutical composition of the present invention to a subject.

The pharmaceutical composition, liver disease, prevention, treatment, and the like are as described in other embodiments.

As used herein, the term “subject” refers to all animals that have or may develop liver disease, and may mean all mammals, including dogs, cows, horses, rabbits, mice, rats, chickens, and humans. By administering the pharmaceutical composition of the present invention to a subject suspected of having liver disease, the subject may be effectively treated.

As used herein, the term “administration” means introducing the pharmaceutical composition of the present invention into a subject suspected of having liver disease by any suitable method, and the pharmaceutical composition may be administered through various routes such as parenteral, subcutaneous, intraperitoneal, intrapulmonary, and intranasal routes as long as it can reach the target tissue in terms of administration route. Parenteral injection includes intramuscular, intravenous, intraarterial, intraperitoneal or subcutaneous administration. The formulation may be, but is not limited to, a tablet, trochee, capsule, elixir, suspension, syrup, wafer, intravenous injection, subcutaneous injection, intradermal injection, intramuscular injection, or instillation formulation. The preferred dosage of the pharmaceutical composition of the present application may vary depending on the condition and body weight of the subject, the severity of the disease, the type of drug, and the route and duration of administration.

The pharmaceutical composition of the present invention can be applied to any one as long as it is a subject of which the liver disease is intended to be prevented or treated without particular limitation. For example, the pharmaceutical composition may be used for any non-human animals such as monkeys, dogs, cats, rabbits, guinea pigs, rats, mice, cows, sheep, pigs, and goats, birds, fish, and the like. The preferred dosage of the pharmaceutical composition of the present invention varies depending on the condition and body weight of the subject, the severity of the disease, the type of drug, and the route and duration of administration, but may be appropriately selected by those skilled in the art. For example, the pharmaceutical composition may be administered by oral, rectal or intravenous, intramuscular, subcutaneous, intrauterine intrathecal or intracerebral injection, but the route is not limited thereto.

The suitable total daily used amount of the pharmaceutical composition of the present invention may be determined by a treating physician within the scope of correct medical judgment, and generally an amount of 0.001 mg/kg to 1000 mg/kg, preferably 0.05 mg/kg to 200 mg/kg, more preferably 0.1 mg/kg to 100 mg/kg may be administered once a day or several times a day in a divided manner.

The pharmaceutical composition of the present invention may be administered in a pharmaceutically effective amount.

As used herein, the term “pharmaceutically effective amount” means an amount sufficient to treat a disease with a reasonable benefit/risk ratio applicable to medical treatment, and the effective dosage level may be determined depending on factors including the kind, severity, age, sex of the subject, the type of the disease, the activity of the drug, the sensitivity to the drug, the time of administration, the route of administration and the excretion rate, the duration of treatment, and the drugs used concurrently, and other factors well known in the medical arts. The composition of the present invention may be administered as an individual therapeutic agent or in combination with other therapeutic agents, and may be administered sequentially or simultaneously with conventional therapeutic agents. The composition of the present invention may be administered singly or in multiple doses. It is important to administer the composition in a minimum amount that can afford the maximum effect without causing side effects in consideration of all the above factors, and the amount can be easily determined by those skilled in the art.

### [Mode for Carrying Out Invention]

Hereinafter, the present invention will be described in more detail with reference to Examples. However, the following Examples are merely preferred embodiments for illustrating the present invention and, therefore, are not intended to limit the scope of the present invention thereto. Meanwhile, technical matters not described in this specification can be sufficiently understood and easily implemented by those skilled in the art of the present invention or a similar technical field.

### Reference Example 1: Statistical Analysis

Data of the present invention were expressed as mean±standard deviation, and statistical analysis was performed by one-way ANOVA (Turkey's multiple comparison test) or unpaired t test using Graphpad Prism software. Data of *P<0.05, **P<0.01, and ***P<0.001 were considered statistically significant.

### Reference Example 2: Material Procurement

In the following Examples, experiments were conducted using the following methods and materials.

All primers for quantitative RT-PCR (qRT-PCR) were procured from Bioneer (Daejeon, Korea). Phosphoramidite (DNA and 2′OMe-RNA), albumin (derived from mouse fragment V), IgG (derived from mouse serum), LDL (human low-density lipoprotein, purified), HDL (human high-density lipoprotein, purified), carbon tetrachloride (CCl₄) and olive oil were procured from Sigma Aldrich (MO, USA). 5′-GalNAc-C3 phosphoramidite was procured from Glen Research (VA, USA), and exonucleases I and II and DNAase I were purchased from New England Biolabs (MA, USA). Streptavidin-coated magnetic beads (Dynabeads™ MyOne Streptavidin™ T1) and SYBR green Master Mix for qRT-PCR were procured from Thermo Fisher Scientific (CA, USA). The RNeasy Mini kit was procured from Qiagen (CA, USA), and the cDNA synthesis kit was purchased from Enzynomics (Daejeon, Korea). Antibodies for Western blotting and immunofluorescence imaging were procured from Cell Signaling Technology (MA, USA), Santa Cruz Biotechnology (TX, USA), Abcam (Cambridge, UK), R&D systems (MN, USA), Bethyl Laboratories Inc. (TX, USA), and Biolegend (CA, USA). BALB/c mice (male, weeks old) were purchased from Orient Bio Inc. (Seongnam, Korea). Other buffers, organic solvents, and chemical reagents were procured from Biosesang (Seongnam, Korea), Samchun chemicals (Seoul, Korea), and Sigma Aldrich (MO, USA), respectively.

### Example 1: Construction of Nucleic Acid Nanoparticle (Td), Complex (Chol₃-Td) of Nucleic Acid Nanoparticle and Cholesterol, Complex (ASO@Td) of ASO and Nucleic Acid Nanoparticle, and Complex (ASO@Chol₃-Td) of ASO, Nucleic Acid Nanoparticle, and Cholesterol

All oligonucleotides were purchased from Integrated DNA Technologies (IDT). The respective strands (S1, S2, S3, and S4 for Td; S1-Choi, S2-Chol, S3-Chol, and S4 for Chol₃-Td) of nucleic acid nanoparticles (hereinafter referred to as Td) and complex (Chol-Td) of nucleic acid nanoparticle and cholesterol were mixed and assembled. In the construction of the structure loaded with ASO, S4-ASO was used instead of unmodified S4. The four strands were mixed with TM buffer (10 mM Tris-HCl, 5 mM MgCl₂, pH 8). The mixture was denatured at 95° C. for 5 minutes using a Thermal Cycler (Applied Biosystems, MA, USA), cooled at 4° C. for 1 hour to allow self-assembling of Td, Chol₃-Td, ASO@Td, and ASO@Chol₃-Td, respectively. The sequences of S1, S2, S3, and S4 for Td; and S1-Chol, 52-Chol, S3-Chol, and S4 for Chol₃-Td are shown in Table 1 below.

[Table 1]

TABLE 1

| SEQ | | |
|---|---|---|
| ID | | |
| NO: | Sequence (5′ to 3′) | |
| 1 | S1 | CCAGGCAGTTGAG |
| | | ACGAACATTCCTA |
| | | AGTCTGAAATTTA |
| | | TCACCCG |
| 2 | S2 | CTTGCTACACGAT |
| | | TCAGACTTAGGAA |
| | | TGTTCGACATGCG |
| | | AGGGTC |
| | | CAATACCGACGAT |
| | | TACAG |
| 3 | S3 | GGTGATAAAACGT |
| | | GTAGCAAGCTGTA |
| | | ATCGACGGGAAGA |
| | | GCATGC |
| | | CCATCCACTACTA |
| | | TGGCG |
| 4 | S1-Chol | CCAGGCAGTTGAG |
| | | ACGAACATTCCTA |
| | | AGTCTGAAATTTA |
| | | TCACCCG |
| | | CCATAGTAGACGT |
| | | ATCA-Chol |
| 5 | S2-Chol | CTTGCTACACGAT |
| | | TCAGACTTAGGAA |
| | | TGTTCGACATGCG |
| | | AGGGTC |
| | | CAATACCGACGAT |
| | | TACAG-Chol |
| 6 | S3-Chol | GGTGATAAAACGT |
| | | GTAGCAAGCTGTA |
| | | ATCGACGGGAAGA |
| | | GCATGC |
| | | CCATCCACTACTA |
| | | TGGCG-Chol |
| 7 | S4 | CCTCGCATGACTC |
| | | AACTGCCTGGTGA |
| | | TACGAGGATGGGC |
| | | ATGCTC |
| | | TTCCCGACGGTAT |
| | | TGGAC |
| 8 | S4-ASO | CTCGCATGACTC |
| | | AACTGCCTGGTGA |
| | | TACGAGGATGGGC |
| | | ATGCTC |
| | | TTCCCGACGGTAT |
| | | TGGACTTTTTGTC |
| | | CACCATTAGCACG |
| | | CGmGmG |
| 9 | S4-SC-ASO | CCTCGCATGACTC |
| | | AACTGCCTGGTGA |
| | | TACGAGGATGGGC |
| | | ATGCTC |
| | | TTCCCGACGGTAT |
| | | TGGACTTTTTGGT |
| | | GGTGTTGGTGGTG |
| | | GTmGmG |
| 10 | ASO | GTCCACCATTAGC |
| | | ACGCGGG |
| 11 | Chol-ASO | GTCCACCATTAGC |
| | | ACGCGGG-Chol |
| 12 | GalNAc3- | GalNAc3-GTCCA |
| | ASO | CCATTAGCACGCG |
| | | GG |

* mG is 2′-O-methoxy-RNA.

The cholesterol-linked nucleic acid nanoparticle (Chol-Td) was constructed so as to be 20-mer double-stranded per side by assembling a 63-mer DNA strand with cholesterol linked at the 3′-end and a 63-mer DNA strand with no cholesterol linked thereto as described above.

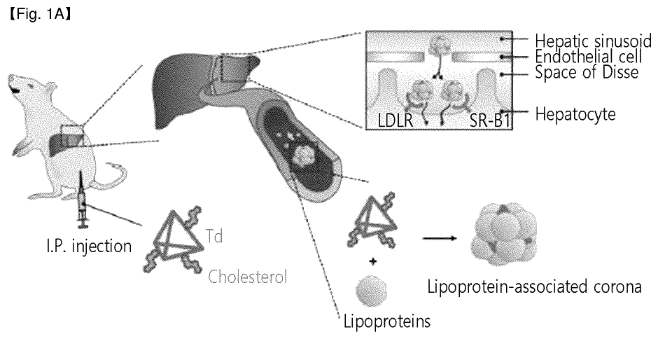

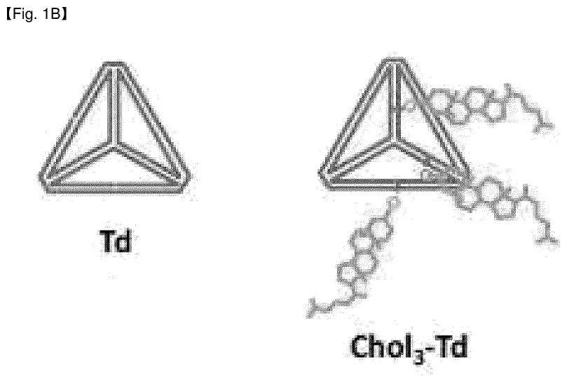

The number of cholesterol linkages can be appropriately changed by adjusting the number of cholesterol-linked strands (for example, 0 to 3 cholesterol molecules), but as an example, a complex (hereinafter referred to as Chol₃-Td) in which three cholesterol molecules were linked to a nucleic acid nanoparticle was constructed (FIG. 1B).

### Example 2: Action Mode Analysis of Complex Containing Nucleic Acid Nanoparticle and Cholesterol

It was confirmed that a protein corona was formed to move the nucleic acid nanoparticle to hepatocytes in situ in a case where a complex containing a nucleic acid nanoparticle and cholesterol was intraperitoneally injected into a subject.

2-1: Construction of Serum Protein-Bound Td and Chol-Td

In order to construct a serum protein-bound structure, the assembled Td and Chol₃-Td were mixed with a serum protein (LDL, HDL, albumin, or IgG) at a molar ratio of Td (or Chol₃-Td):serum protein=1:8 (1×) in PBS and incubated at 37° C. for 1 hour.

2-2: Gel Electrophoresis Analysis

Hereinafter, the gel electrophoresis in the Examples was performed by way of the following analysis method.

Each assembled structure was verified by native PAGE (6%). Electrophoresis was performed in 0.5× Tris-borate-EDTA (TBE), 100 V, and 4° C. for 50 minutes, and Cy5 was labeled at the 5′ end of the S4 strand for visualization. After electrophoresis, the gels were imaged using an iBrightFL1000 (Thermo Fisher Scientific).

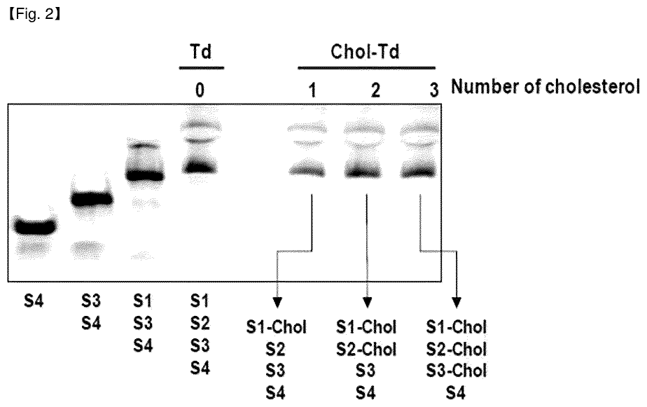

As a result of gel electrophoresis of Td, Chol₁-Td. Chol₂-Td, and Chol₃-Td, all results of these were similar (FIG. 2).

2-3: Dynamic Light Scattering (DLS) Analysis

Hereinafter, the dynamic light scattering in the Examples was performed by way of the following analysis method. The hydrodynamic sizes of unmodified, ASO-loaded Td, and Chol₃-Td were measured using a Zetasizer (Malvern Instruments, Worcestershire, UK). The concentration of the sample used for DLS analysis was set to 250 nM. In order to analyze serum protein binding properties, a mixture of Tds (200 nM) and serum proteins (1.6 μM) were used for measurement.

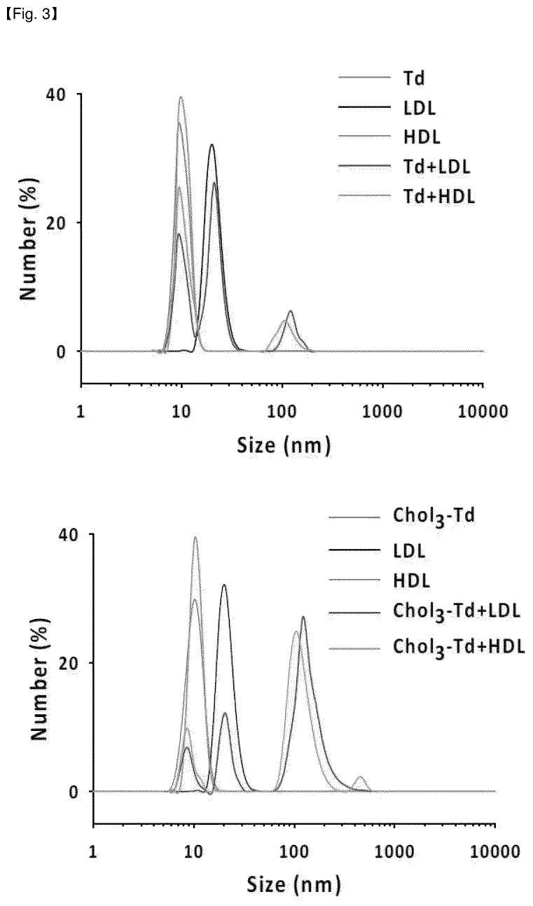

As a result, similar to gel electrophoresis, it was confirmed that the sizes of Chol₃-Td and Td are similar as Chol₃-Td (10 nm) has a slightly larger hydrodynamic size than Td (9 nm) (FIG. 3).

Meanwhile, it was observed that the size of the nanoparticles significantly increases (90 nm to 150 nm) when the DNA nanoparticles are incubated with LDL or HDL (FIG. 3). Increases in the size of a relatively large number of Chol₃-Td compared to Td were observed, and this shows that cholesterol of Chol₃-Td increases lipoprotein avidity.

2-4: Atomic Force Microscopy (AFM) Analysis

Hereinafter, atomic force microscope analysis in the Examples was performed by way of the following method. The size and shape of Td and Chol₃-Td were observed under an atomic force microscope (AFM; XE-100, Park Systems, Suwon, Korea). Prior to the sample treatment, the silicon wafer (Si-Wafer) was coated with NiCl₂ (2 mM) for 1 minute. After the surface of the Si-wafer was washed with distilled water (DW), the Td or Chol₃-Td (50 μL, 10 nM) in the TM buffer was dropped on the wafer and incubated for 1 minute, and treated with Mg(OAc)₂ (25 μL, 2 mM). In the case of serum protein (80 nM)-bound Td and Chol₃-Td (10 nM), the samples were not treated with NiCl₂ but dropped on mica. AFM images were acquired in the non-contact mode using non-contact cantilever (PPP-NCHR, Park Systems) and analyzed with XEI software (Park Systems).

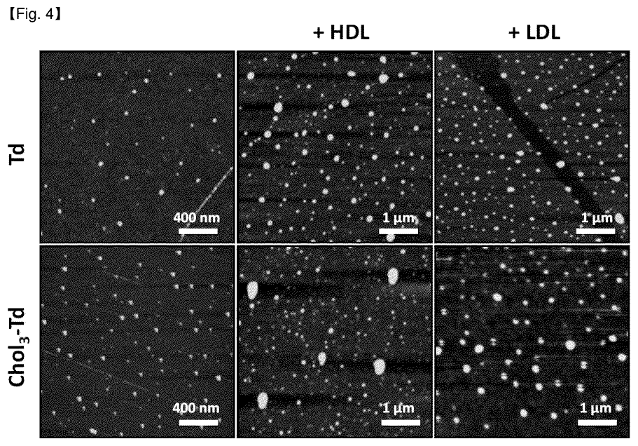

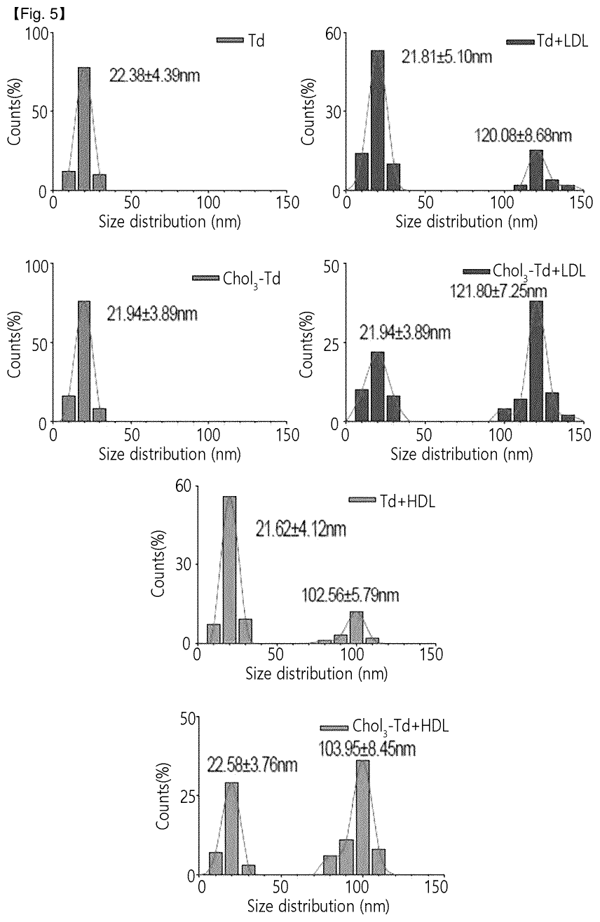

As a result, as in the dynamic light scattering data, it was confirmed that the lipoprotein complex formed by Chol₃-Td is larger in size than the complex formed with Td in the atomic force microscopy (AFM) images as well (FIGS. 4 and 5).

In addition, even if the size of the protein corona is not significantly different from the size of the liver sinusoid endothelial fenestrae (100 nm to 140 nm), the nucleic acid nanoparticle and lipoprotein structures may be modified, and thus the protein corona may easily pass through the liver sinusoid endothelial fenestrae.

2-5: Stability of Lipoprotein (LDL/HDL)-Bound Td and Chol₃-Td with Respect to Nuclease

Lipoprotein-bound Td and Chol₃-Td (Cy5-labeled, 1 μM) were incubated with Exonucleases I and III (Exonuclease I: 20 units, Exonuclease III: 100 units) and DNase I (4 units) at 37° C. for 0, 1, 4, 7 and 24 hours. At each time point, the reaction was completed by addition of loading buffer (95% formaldehyde, 0.5 M EDTA) and heating at 95° C. for 10 minutes. Thereafter, the samples were analyzed by 10% denaturing PAGE, which was performed in 0.5×TBE at 180 V for 1 hour. Gels were visualized using iBright FL1000.

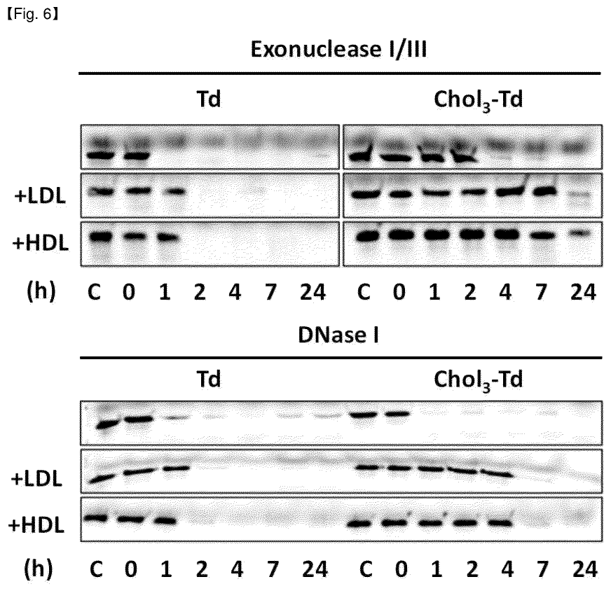

As a result, it was confirmed that the lipoprotein corona imparts in vivo stability by substantially preventing DNA nanoparticles from being degraded by nucleases (FIG. 6).

From the results, it was confirmed that the complex containing a nucleic acid nanoparticle and cholesterol forms a protein corona in the body, and this increases its stability in the body.

### Example 3: Analysis of Protein Type Contained in Protein Corona

3-1: Pull-Down Assay of Serum Protein Bound to Td and Chol-Tds

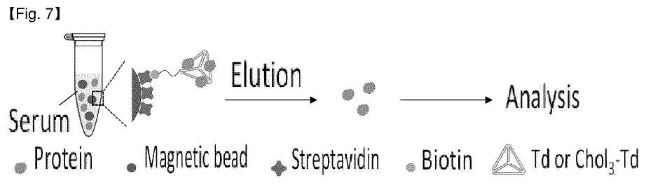

In order to analyze serum proteins absorbed by nucleic acid nanoparticles, serum proteins linked to Chol₃-Td immobilized on magnetic beads were pulled down and analyzed by SDS-PAGE. A schematic diagram of the pull-down assay is illustrated in FIG. 7, and the pull-down assays in the Examples below was performed by way of the following analysis method.

Streptavidin-coated magnetic beads (20 μL) were washed three times with PBS (100 μL, 1×). In order to remove proteins non-specifically linked to the magnetic beads, the washed beads were redispersed in binding buffer (50 μL, 2×PBS) with mouse serum (50 μL, Sigma-Aldrich) at 37° C. for 1 hour. Biotinylated-Td (1 μM) containing 0 to 3 cholesterol molecules was immobilized on streptavidin-coated magnetic beads and mixed with the protein supernatant (100 μL). The mixture was incubated at 37° C. for 1 hour. Magnetic beads were separated from unbound serum proteins and washed three times with 1×PBS. The magnetic beads were redispersed in loading buffer (50 mM Tris-HCl, pH 6.8, 2% SDS, 6% (v/v) glycerol, 2 mM DTT, 0.01% (w/v), Bromophenol Blue) and incubated at 95° C. for 10 minutes. Proteins bound to Tds-immobilized magnetic beads were analyzed by 5% to 12% SDS-PAGE and stained with Coomassie blue. Gel images were acquired using iBright FL1000.

In order to detect the level of specific serum proteins (ApoA-1 as HDL marker, ApoB, IgG and albumin as LDL marker) in the pulled-down sample, Western blotting analysis was performed. Briefly, pulled-down samples were run on 5% to 12% SDS-PAGE, transferred to polyvinylidene difluoride (PVDF) membranes, and incubated overnight at 4° C. in a solution (5% BSA/TBST) containing an antibody (anti ApoA-1, 1:1000, Thermo Fisher Scientifics or anti ApoB, 1:1000, Santa Cruz Biotechnology). Thereafter, these were incubated with HRP-conjugated secondary antibody (horseradish peroxidase-conjugated secondary antibody; 1:10,000 in 5% skim milk/TBST, Cell Signaling Technology) at room temperature for 1 hour. In order to detect albumin and IgG, HRP-conjugated-primary antibody (anti-mouse IgG-HRP or anti-mouse albumin-HRP, 1:10,000 in 5% skim milk-TBST, Bethyl Laboratories Inc.) was used. Finally, protein bands were visualized using Super Signal™ West Pico Chemiluminescent (Thermo Fisher Scientific) and imaged using iBright FL1000.

In order to determine the dissociation constant (K_{d}) between Td (or Chol₃-Td) and a protein (albumin, IgG, LDL, or HDL), a similar method was introduced. Briefly, streptavidin-coated magnetic beads (10 μL) were incubated with Td or Chol₃-Td (20 pmol) in binding buffer (1×PBS, 100 μL) at room temperature for 30 minutes, and washed three times with 1×PBS. Td or Chol₃-Td-immobilized beads were incubated with a protein at various contents (80 pmol, 160 pmol, 400 pmol, and 800 pmol) in 1×PBS (100 μL) at 37° C. for 1 hour. After unbound proteins were removed, the beads were washed with 1×PBS. In order to elute proteins that are bound to Td or Chol₃-Td, the beads were redispersed in 20 μL of loading buffer and denatured at 95° C. for 10 minutes. The eluted samples were run on 5% to 10% SDS-PAGE and stained with Coomassie blue. For quantitative analysis of protein, various control contents of protein (1 μg, 2 μg, 5 μg, and 10 μg of albumin, IgG, and HDL, and 5 μg, 10 μg, 20 μg, and 50 μg of LDL) were loaded on SDS-PAGE. The contents of bound proteins were measured by band intensities quantified using Image J software (National Institutes of Health, MD, USA). The dissociation constant was calculated by curve-fitting with the ligand binding formula in SigmaPlot software.

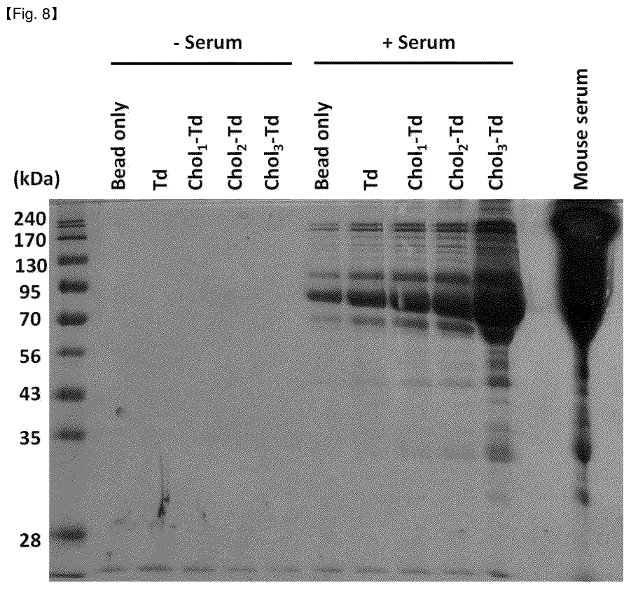

As a result of SDS-PAGE and Coomassie blue staining, it was confirmed that the amount of serum protein absorbed by Td increases depending on the number of cholesterol molecules bound to Td (FIG. 8).

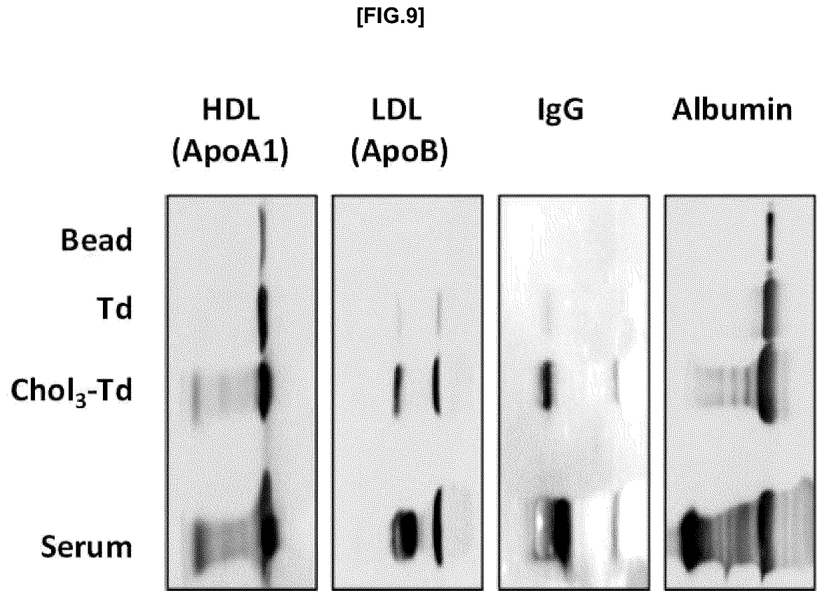

As a result of Western blotting analysis of the pulled-down proteins, lipoproteins and other major serum proteins such as albumin and immunoglobulin (IgG) were also absorbed by Chol₃-Td (FIG. 9).

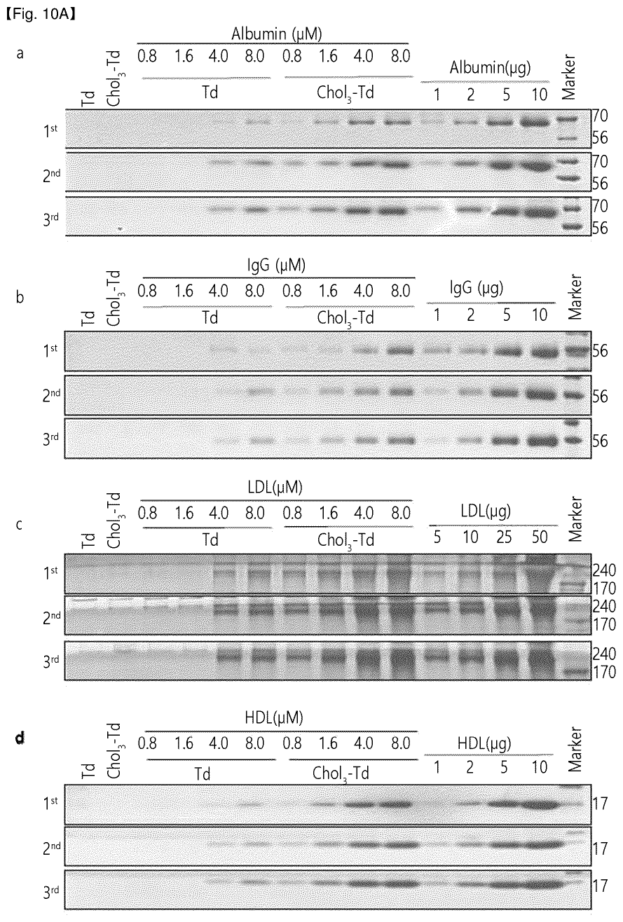

As a result of dissociation constant (K_{d}) analysis determined by SDS PAGE, the trivalent cholesterol linkage generally increases the avidity of serum proteins to DNA nanoparticles, and facilitates binding to lipoproteins compared to albumin and IgG (FIGS. 10A and 10B).

For reference, in FIG. 10, K_{d} of albumin, IgG, LDL, and HDL in Chol₃-Td is the average of the dissociation constants acquired through three independent experiments, standard deviations (SD) are indicated in parentheses, and N.D. means that the value is not determined due to weak binding.

3-2: Proteomic Analysis

Since lipoproteins, albumin, and IgG are not the only constituents of the protein corona of serum DNA nanoparticles, in order to confirm other constituents of the protein corona as well, proteins pulled down from Td and Chol₃-Td were analyzed proteomically based on LC-MS/MS (liquid chromatography-tandem mass spectrometry). The proteomic analysis method was used as follows, and the proteomic analysis in the Examples below was performed by way of the following method.

Proteins pulled down from beads. Td, and Chol₃-Td in duplicate were separated based on the molecular weight by 10% SDS-PAGE. After the gel was stained with Coomassie blue and de-stained with distilled water, the stained gel for each sample lane was separated into eight slices, and proteins contained in each gel slice were used for trypsin digestion. The proteins were first reduced using 10 mM DTT in 25 mM NH₄HCO₃ at 56° C. for 1 hour, alkylated with iodoacetamide in 25 mM NH₄HCO₃ at 25° C. for 1 hour in the dark, and then subjected to trypsin digestion overnight. Peptides were extracted using 67% acetonitrile (ACN)/5% formic acid (FA) in water, dried in a SpeedVac, and then redispersed with 20 μL of 0.4% acetic acid. For mass spectral analysis, 13.5 μL of each sample was injected into a reversed-phase Magic C18AQ column (15 cm×75 μm) in Eksigent MDLC system (Eksigent Technologies, CA, USA). The operating flow rate was set to 350 nL/min, and the following progressive conditions were set: 100% buffer A (100% water containing 0.1% FA) and 0% buffer B (100% ACN containing 0.1% FA) at 0 min, 0%-8% B from 0 min to 5 min, 8%-30% B from 5 min to 85 min, 30% 70% B from 85 min to 90 min, 70% B from 90 min to 100 min. 70%-2% B from 100 min to 110 min, and 2% B from 100 min to 120 min. The nano HPLC system was connected to an LTQ XL-Orbitrap mass spectrometer (Thermo Fisher Scientific, MA, USA). The spray voltage was set to 2.5 kV, and the temperature of the heated capillary was set to 250° C. Survey full-scan mass spectrometry spectra (MS; 300-1800 m/z) with 1 micro scan at a resolution of 60,000 were acquired to allow viewing of the preview mode for precursor selection and charge-state determination. Tandem mass (MS/MS) spectra were acquired for the 10 most intense ions in the ion trap and under the following conditions: isolation width, 2 m(z; normalized collision energy, 35%; dynamic exclusion duration, 360 seconds. During data-dependent acquisition, precursors with +1 charge and precursors with unassigned charge states were discarded. Each LC-MS/MS file was searched against the SwissProt mouse database (July 2021) with 17225 entries using Proteome Discoverer software (version 2.4, Thermo Fisher Scientific, Bremen, Germany). The search criterion was set at a mass tolerance of 15 ppm for MS data and 0.5 Da for MS/MS data using the fixed modification (+57.021 Da) of carbamidomethylation of cysteine and the variable modification (+15.995 Da) of methionine oxidation. The false discovery rate (FDR) was set to 0.01 for peptide and protein identification. All proteins were identified as two or more unique proteins. For label-free quantification, the normalized abundance values of protein for each sample were acquired from the peak area normalized by the total peptides using Minora algorithm-based label-free quantification in Proteome Discoverer 2.4. The normalized abundance values acquired by label-free quantification were statistically analyzed using Perseus software (1.6.14.0). The normalized presence values were log-transformed, and missing values were replaced with values calculated from a normal distribution with a width of 0.3 and a downshift of 1.8. Proteins showing statistical significance among the samples pulled down in beads, Td, and Chol₃-Td were acquired by ANOVA comparison of log 2 (normalized abundance) values from two replicates of each sample. The resulting p-values were adjusted for FDR using the Benjamini and Hochberg method. FDR-adjusted P-values <0.05 were statistically significant. For hierarchical clustering of proteins showing statistically significant changes (>1.5-fold, FDR-adjusted P-value <0.05) among samples pulled down from beads, Td, and Chol₃-Td, the abundance values were normalized using z-scores, and column and row clustering was attempted based on Euclidean distance using Perseus (1.6.14.0) and the average linkage method. GO (gene ontology) functional classification was analyzed using DAVID software (http://david.abcc.ncifcrf.gov). In order to identify GO terms significantly enriched in proteins showing significant differences in samples pulled down from Chol₃-Td compared to samples pulled down from Td, GO term over-representation analysis was performed. A cutoff value of FDR-adjusted P-value <0.05 was used to report a functional category as significantly overrepresented.

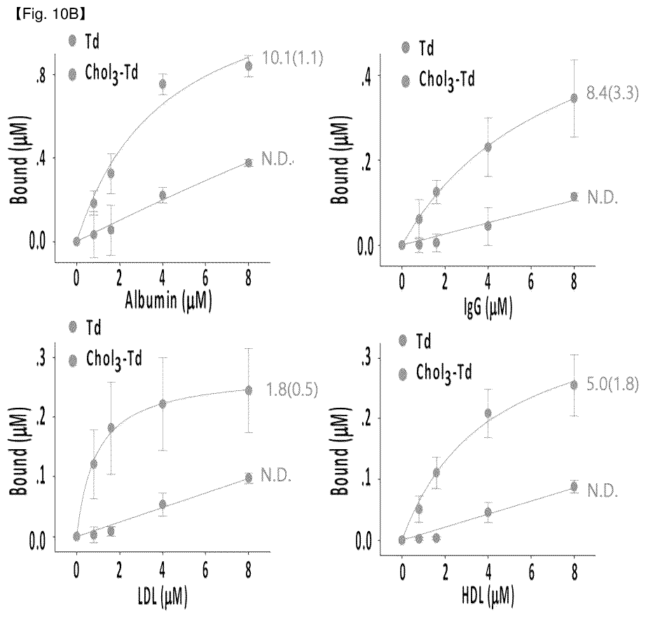

As a result, 105, 186, and 232 proteins were identified in the samples pulled down from beads, Td, and Chol₃-Td, respectively, and 97 proteins were confirmed to be common among these. As a result of label-free quantitative analysis, statistically significant 197 proteins were found (false discovery rate (FDR)-adjusted P-value <0.05). The 197 proteins were hierarchically clustered, and it was confirmed that the number of proteins further increases by Chol₃-Td pull-down than by bead or Td pull-down (FIG. 11; heat map).

For reference, in FIG. 11, rows represent each protein and columns represent two replicates of protein samples pulled down from beads, Td and Chol₃-Td. Hierarchical clustering of 197 proteins was performed using Perseus software (1.6.14.0) for log-transformed normalized abundance values after z-score normalization of the data.

When relative normalized abundance values were calculated from 197 proteins pulled down from Chol₃-Td or Td, the levels of 191 proteins have significantly increased in Chol₃-Td compared to Td of protein corona (>1.5-fold, FDR-adjusted P-value <0.05). The abundance value data show that 190 out of 191 proteins bind more firmly to Chol₃-Td than to Td.

Thereafter, the list of proteins known in previous proteomic studies of isolated HDL and LDL particles and phospholipid-containing particles (Davidson, W. S. “The HDL Proteome Watch”, 2021, http://homepages.uc.edu/˜davidswm/HDLproteme.html; Davidson, W. S. “The LDL Proteome Watch”, 2021, http://homepages.uc.edu/˜davidswm/LDLproteome.html; and Gordon, S. M.; Li, H.; Zhu, X.; Shah, A. S.; Lu, L. J.; Davidson, W. S. A Comparison of the Mouse and Human Lipoproteome: Suitability of the Mouse Model for Studies of Human Lipoproteins. J. Proteome Res. 2015, 14, 2686-2695) was compared with 190 Chol₃-Td-preferred proteins to search the number of proteins associated with lipoproteins among the proteins firmly bound to Chol₃-Td. Among the 190 proteins, 105 proteins and 27 proteins had corresponding human analogues compared to human HDL and LDL proteomes, respectively, and 84 proteins were similar to mouse lipid-associated proteins (FIG. 11).

From the results, it was confirmed that 66% (125/190) of the proteins exhibiting high affinity to Chol₃-Td are associated to lipoproteins.

### Example 4: Evaluation of Ability to Deliver Drug to Hepatocyte of Complex Containing Nucleic Acid Nanoparticle and Cholesterol

4-1: Animal Imaging Analysis

Hereinafter, the animal imaging in the Examples was performed by way of the following analysis method.

Experiments using live animals were conducted in conformity with relevant laws and institutional guidelines (2020-005). For in vivo imaging, Cy5-labeled DNA structures (Td and Chol₃-Td for healthy mouse biodistribution; ASO and ASO@Chol₃-Td for liver fibrosis induced mouse biodistribution) (1 μM, 200 μL) were intraperitoneally administered to BALB/c mice. Fluorescence images were acquired using IVIS imaging Spectrum System (emission at 670 nm/excitation at 620 nm) and analyzed with IVIS Living Imaging 3.0 software.

In this Example, in order to evaluate that Chol₃-Td could form a lipoprotein-associated protein corona, Cy5-labeled Chol₃-Td (200 pmol) was intraperitoneally administered to BALB/c mice, and the in vivo fluorescence intensity was measured to determine the ability of DNA nanoparticles based on protein corona to be delivered to the liver.

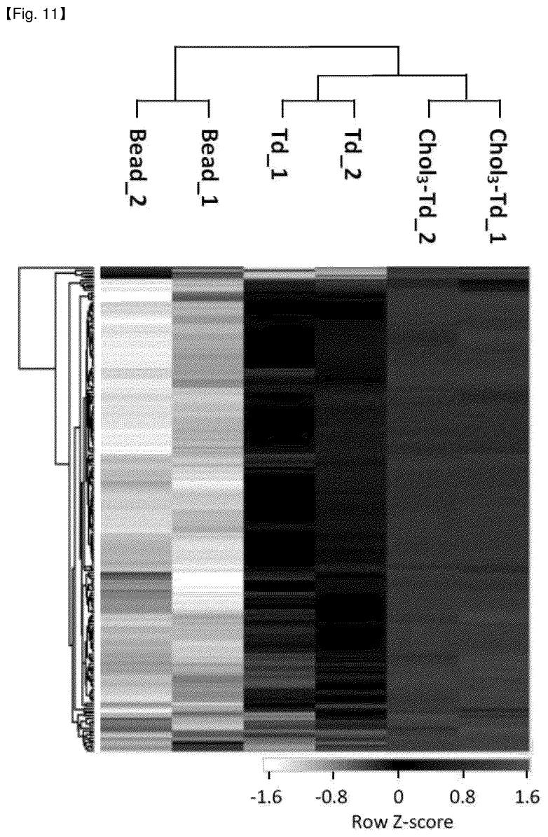

When the total in vivo intensity was highest, major organs were harvested from the mice 2 hours after injection, and the distribution of DNA nanoparticles was observed based on the ex vivo fluorescence intensity. From ex vivo imaging of major organs, it was confirmed that Chol₃-Td is distributed in the liver at a very high level (FIGS. 12 and 13).

As a result, it was confirmed that the liver-targeting ability of cholesterol-linked Chol₃-Td is significantly increased compared to Td, and the renal distribution for renal elimination is decreased.

4-2: Organ Lysate Analysis

Hereinafter, the organ lysate analysis in the Examples was performed by way of the following analysis method.

As in Example 4-1, 2 hours after injection, the mice were sacrificed, and organs were harvested. The cut tissue was homogenized under liquid nitrogen and dissolved in RIPA buffer (Cell Signaling Technology). The lysed tissue was centrifuged (12,000 rpm, 10 min, 4° C.), and the supernatant of each sample was analyzed using a fluorescence spectrophotometer (F7000, Hitachi, Tokyo, Japan). After excitation at 640 nm, maximum emission intensity was acquired at 680 nm that was determined after scanning in the range of 650 nm to 750 nm.

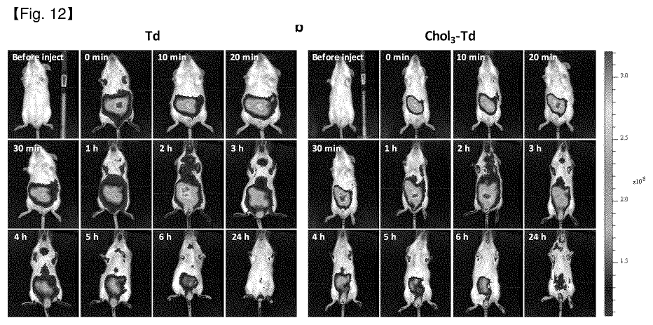

For quantitative analysis of organ distribution of Chol₃-Td, ID %/g of DNA nanoparticles in each organ was evaluated by measuring the average Cy5 intensity of each tissue lysate obtained through homogenization (FIG. 14).

As a result, it was confirmed that Chol₃-Td reaches about 30 ID %/g in the liver and is thus remarkably more distributed in the liver than in the kidney, but Td is remarkably more distributed in the kidney than in the liver (FIG. 14).

4-3: Analysis of Colocalization of Td (or Chol₃-Td) and Cell Marker in Healthy Mouse Liver Tissue

Hereinafter, the colocalization analysis in the Examples was performed by way of the following analysis method.

In order to analyze the localization of Td and Chol₃-Td in hepatocytes, endothelial cells, and macrophages, each cell was immuno-stained with a cell-specific antibody, and the fluorescence signal was analyzed.

The livers were obtained from mice 2 hours after administration of Cy5-labeled Td or Chol₃-Td to the mice. Cut liver tissues were embedded in compound (Leica Biosystems, Nussloch, Germany) at the optimal cutting temperature (OCT) and completely frozen at −80° C. The frozen tissue blocks were cut in a thickness of 15 μm at Lab Core Incorporation (Seoul, Korea). The frozen sections were immunostained overnight at 4° C. with HNF-4α (for hepatocytes), F4/80 (for macrophages), or CD-31 (for endothelial cells) primary antibodies (1:200, Santa Cruz Biotechnology). The sections were washed and incubated with an FITC-labeled secondary antibody (1:1000, Cell Signaling Technology) at room temperature for 1 hour. After the washing step, slides were treated with DAPI-containing mounting solution (Abcam), and imaging was performed using a confocal microscope (LSM 800). Images were acquired from 12 random parts per sample and quantified using Image J software 1.45 (National Institutes of Health). The level of localization was calculated from the colocalized signal (FITC and Cy5, yellow in the image) versus the structure (Cy5, red in the image).

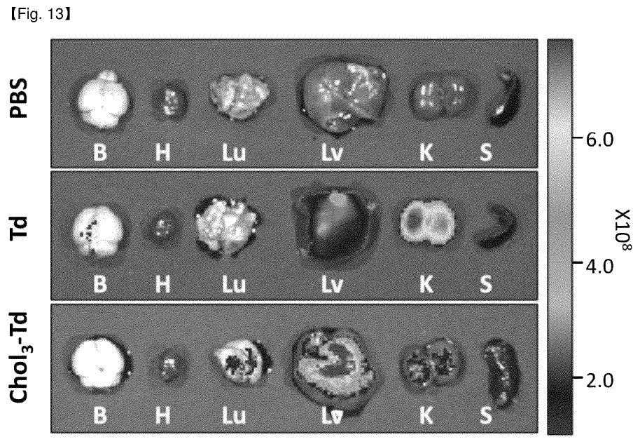

In order to observe the cellular distribution of DNA nanoparticles in the liver, the colocalization of DNA nanoparticles in hepatocytes, endothelial cells, and macrophages of liver sections was imaged, and as a result, Cy5-labeled DNA nanoparticles (red) were absorbed by liver cells visualized with an FITC-labeled antibody specific for each cell type. The nucleic acid image was stained with DAPI (blue) and inserted (FIG. 15).

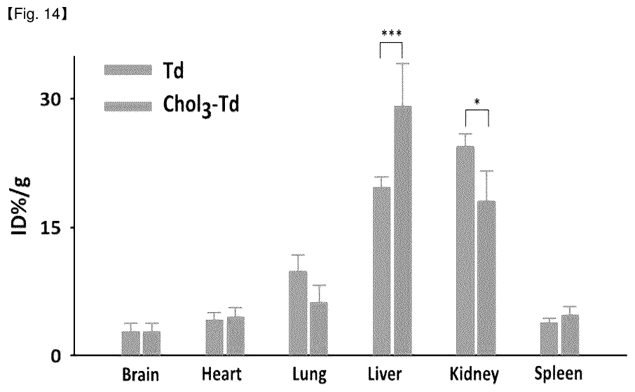

The Td and Chol₃-Td colocalization rates (%) were examined in each cell type, as a result, hepatocytes absorbed Chol₃-Td more efficiently than Td, but endothelial cells and macrophages absorbed Chol₃-Td and Td at similar levels (FIGS. 15 and 16).

It is expected that the greater distribution of Chol₃-Td in hepatocytes is because cholesterol linkage induces a lipoprotein-associated protein corona.

4-4: Cellular Uptake Study

Hereinafter, the cellular uptake study in the Examples was conducted by way of the following analysis method.

In order to analyze the cellular uptake of Td and Chol₁₋₃-Td, HepG2, Raw264.7 and bEnd.3 cells (Korean Cell Line Bank, Seoul, Korea) were seeded in 24-well plates (5×10⁴ cells/well). After 24 hours, cells were washed two times with PBS and incubated with Cy5-labeled Td (or Chol₁₋₃-Td; final concentration=100 nM) or protein (LDL, HDL, IgG, or albumin)-bound DNA structures in serum-free DMEM (Welgene, Gyeongsan, Korea) in a 5% CO₂ incubator at 37° C. After 6 hours, the cells were washed three times with PBS, redispersed in ice-cold PBS (500 μL), and analyzed using a flow cytometer (Guava, Millipore, MA, USA). Samples of at least 10,000 cells were analyzed in triplicate. The Δ uptake rate was calculated as follows.

ΔUptake rate (%)=uptake rate in presence of serum protein (%)−uptake rate in absence of serum protein (%)

As an evaluation result of the cellular uptake of Chol₃-Td in hepatocytes (HepG2), the cellular uptake increased by about 8% to 9% by pre-incubation with lipoproteins (LDL and HDL) and by about 3% to 5% by pre-incubation with other major serum proteins, such as albumin and IgG. In addition, the increase in cellular uptake of DNA nanoparticles was positively correlated with the number of linked cholesterol molecules.

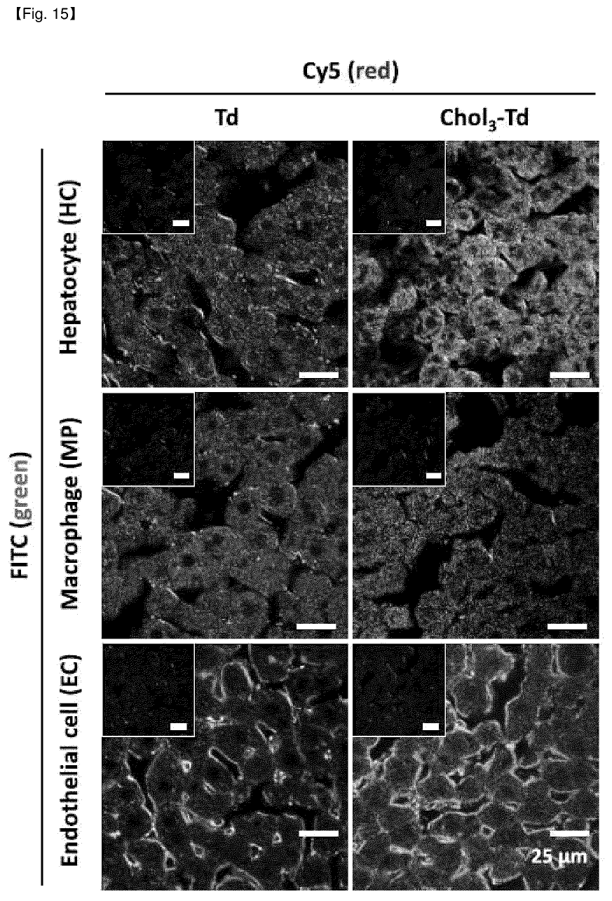

However, uptake of DNA nanoparticles into macrophages (Raw264.7) and endothelial cells (bEnd.3) was not significantly affected by cholesterol linkage. Such results correspond to the in vivo distribution of Td and Chol₃-Td in liver macrophages and endothelial cells (FIGS. 16 and 17).

From the results, it was confirmed that Chol₃-Td can be specifically distributed in the liver and hepatocytes through the cholesterol transport system.

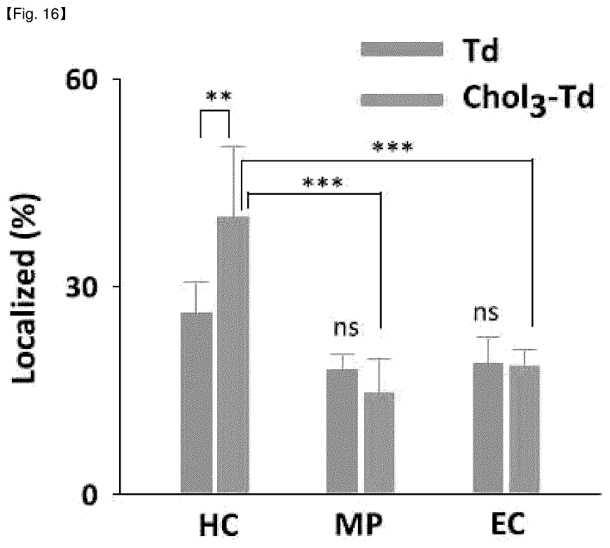

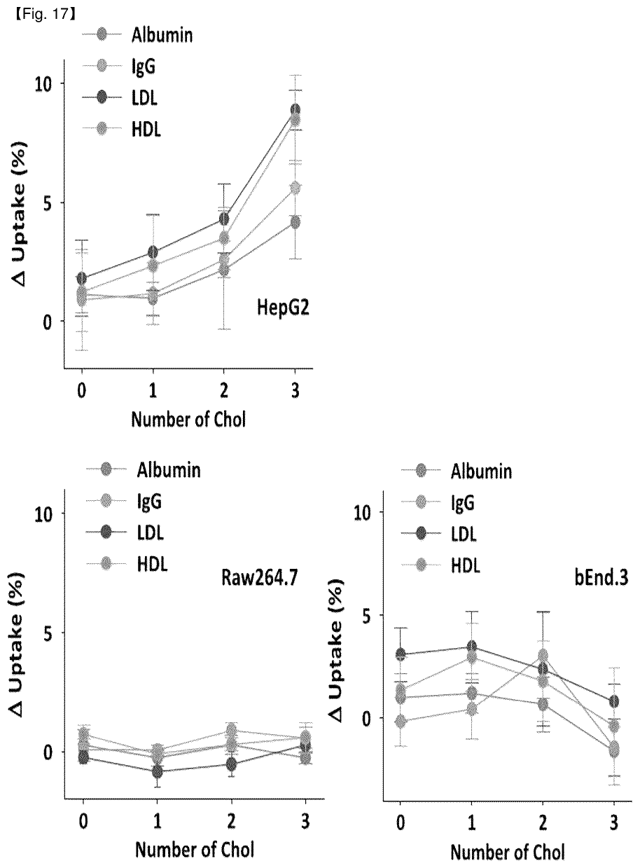

In addition, the hepatocyte uptake efficiency of Chol₃-Td bound to HDL or LDL greatly decreased in the presence of anti-SR-B1 or anti-LDLR antibodies. Meanwhile, Td was not affected by the antibodies (FIGS. 18 and 19). Such results indicate that hepatocyte-preferred uptake of Chol₃-Td is mediated by SR-B1 and LDLR, and these are membrane receptors deeply involved in hepatocyte uptake of HDL and LDL particles.

This result shows that lipoproteins assembled by cholesterol linkages form a protein corona, which is important for efficient internalization of Chol₃-Td into hepatocytes, in the liver.

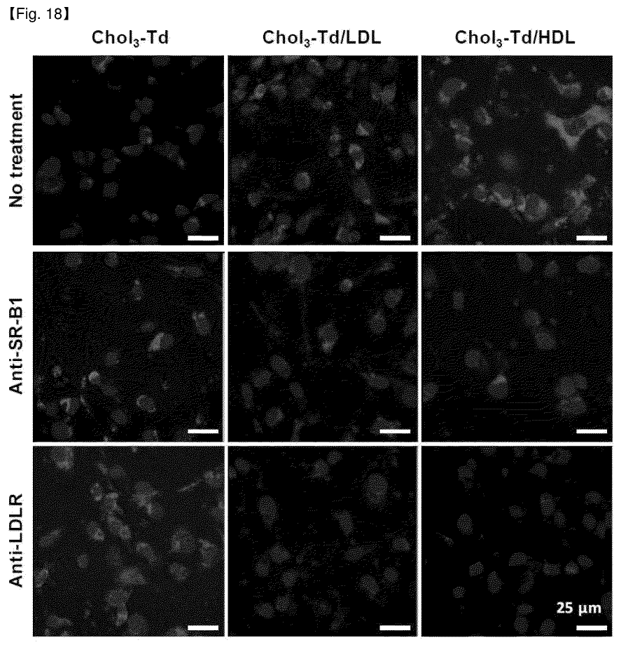

Moreover, it was confirmed that hepatocyte survival is not affected by the treatment with DNA nanoparticles, and the present invention does not exhibit cytotoxicity (FIG. 20).

From the results, it was confirmed that the complex containing a nucleic acid nanoparticle and cholesterol invention does not exhibit cytotoxicity and has excellent liver tissue and/or hepatocyte specific drug delivery ability.

### Example 5: Evaluation of Drug Delivery Ability of Complex Containing Nucleic Acid Nanoparticle, Cholesterol, and Pharmaceutically Active Ingredient to Hepatocyte

In order to treat liver disease, the ability of a complex containing not only a nucleic acid nanoparticle and cholesterol but also a pharmaceutically active ingredient to deliver the pharmaceutically active ingredient to the liver was evaluated. To this end, an antisense oligonucleotide (ASO), a pharmaceutically active ingredient, was linked to Chol₃-Td to evaluate the drug delivery ability for liver fibrosis treatment.

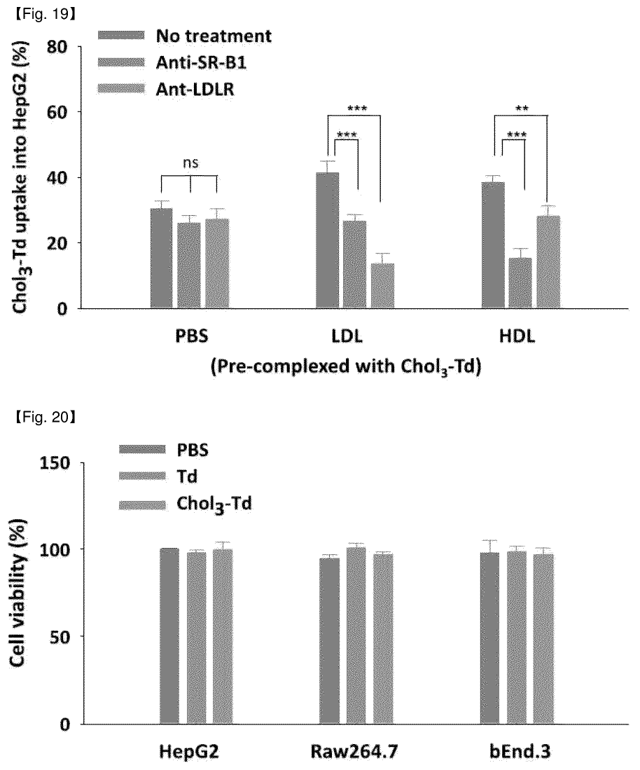

Structures (hereinafter referred to as ASO@Td and ASO@Chol₃-Td, respectively) in which Td and Chol₃-Td were loaded with ASO targeting TGF-β1 mRNA, the most important gene in liver fibrosis, are schematically illustrated in FIG. 21. These were prepared by linking ASO to the 3′ end of the S4 strand of Td shown in Table 1.

5-1: Quantitative RT-PCR Analysis (Analysis of TGF-β1 mRNA, Important Gene for Liver Fibrosis)

In order to investigate ASO-mediated gene regulation, HepG2 cells were seeded in 12-well plates (2×10⁵), and treated with ASO single strands (ASO and Chol-ASO), ASO-loaded structures (ASO@Td, ASO@Chol₃-Td, and ASO-SC@Chol₃-Td), or other controls (Td and Chol₃-Td). Total RNA was extracted from the cells using RNeasy Mini kit (Qiagen) 24 hours after transfection, and their concentration and purity were measured using Nanodrop (Thermo Fisher Scientific). For cDNA synthesis, 2 μg of total RNA and random hexamers were used. 2×SYBR Green master Mix (Thermo Fisher Scientific) and TGF-β1 primer pair (SEQ ID NO: 13 and SEQ ID NO: 14; Forward: TACCTGAACCCGTGTTGCTCTC, Reverse: GTTGCTGAGGTATCGCCAGGAA) or GAPDH primer pair (SEQ ID NO: 15 and SEQ ID NO: 16; Forward: AGAGCTACGAGCTGCCTGAC, Reverse: AGCACTGTGTTGGCG TACAG) were mixed with the synthesized cDNA, and amplification was performed using StepOnePlus real-time PCR systems (Applied Biosystems). The amount of amplified TGF-β1 was normalized to the amount of GAPDH amplified based on the 2^{-ΔΔCt} method. In order to measure the level of TGF-β1 mRNA in vivo, total RNA was isolated from homogenized liver tissue and analyzed in the same manner as above. The primer sequences for in vivo samples are as follows; TGF-β1 (SEQ ID NO: 17 and SEQ ID NO: 18; Forward: CACTCCCGTGGCTTCTAGTG, Reverse: GCGGGTGACCT CTTTAGCAT) and GAPDH (SEQ ID NO: 19 and SEQ ID NO: 20; Forward: GCCATGTACGTAGCCATCCA, Reverse: ATGGCGTGAGG GAGAGCATA).

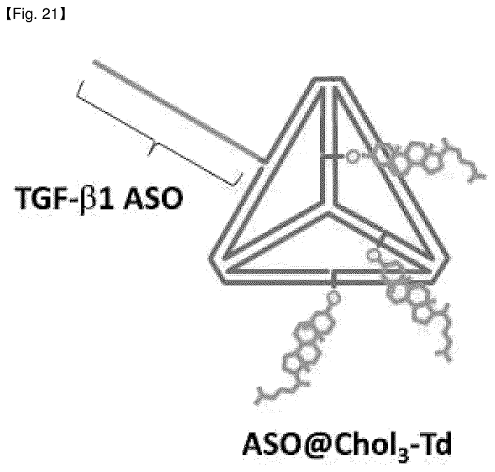

As a result, it was confirmed that the level of TGF-β1 mRNA significantly decreases when HepG2 is treated with ASO@Chol₃-Td, and ASO@Td is not as efficient as ASO@Chol₃-Td (FIG. 22). In the case of ASO, target gene silencing is determined by its sequence, and thus a decrease in the target gene has not been observed in Chol₃-Td loaded with scrambled ASO (ASO-SC@Chol₃-Td) or in structures containing only drug carriers (Td and Chol₃-Td). In addition, free ASO and cholesterol-linked ASO (Chol-ASO) did not significantly affect target gene silencing.

5-2: Western Blotting Analysis (TGF-β1 Protein Expression Analysis)

Proteins (20 μg) derived from cell or tissue lysates were isolated by 10% SDS-PAGE, transferred to PVDF membranes, and incubated with anti-TGF-β antibody (1:1000, Cell Signaling Technology for cell lysate and Santa Cruz Biotechnology for liver lysate), anti-collagen I antibody (1:1000, Abcam), or anti-GAPDH antibody (1:1000, Cell Signaling Technology) at 4° C. overnight. Thereafter, the cells were incubated at room temperature for 1 hour with an HRP-conjugated secondary antibody (1:10,000, Santa Cruz Biotechnology). After the washing step, protein bands were visualized using a Super Signal West Pico.

As a result, it was confirmed that the decreased target mRNA level lowers the down-response of protein expression as observed in Western blotting, and ASO@Chol₃-Td effectively decreases the expression levels of TGF-β1 mRNA, the most important gene in liver fibrosis, and its protein (FIG. 22).

The results show that the complex, which additionally contains a pharmaceutically active ingredient, can also be delivered to hepatocytes and effectively function as the complex containing a nucleic acid nanoparticle and cholesterol does.

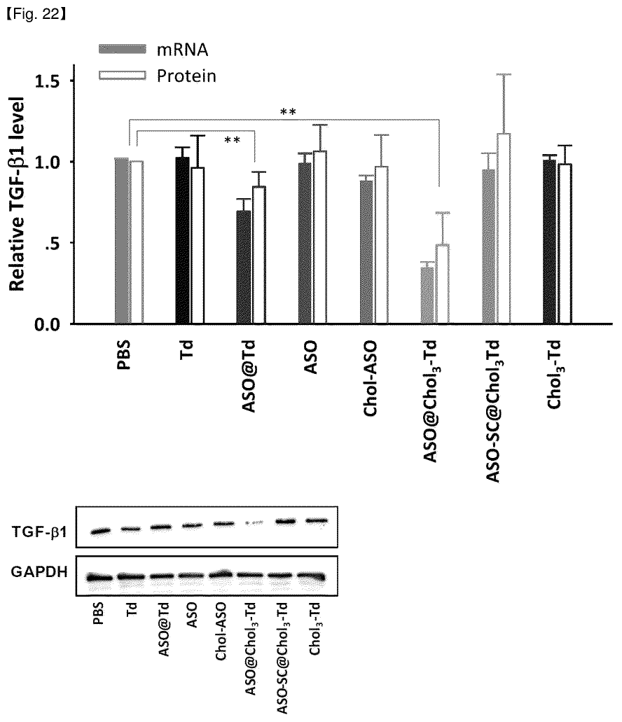

In addition, there is a positive correlation between gene silencing and cellular uptake efficiency (FIG. 23), and this shows that cellular uptake is important for the activity efficiency of intracellular ASO by Td- and cholesterol-mediated nano-agents.

### Example 6: Evaluation of Drug Delivery Ability of Complex Containing Nucleic Acid Nanoparticle, Cholesterol, and Pharmaceutically Active Ingredient in Liver Fibrosis Animal Model

It was evaluated whether a complex containing a nucleic acid nanoparticle, cholesterol, and a pharmaceutically active ingredient could treat liver disease. To this end, it was examined in vivo whether ASO@Chol₃-Td could treat liver fibrosis by increased drug delivery ability.

6-1: Construction of Liver Fibrosis Animal Model

In order to induce liver fibrosis, a mixture (2:8, v/v, 1 mL/kg) of carbon tetrachloride (CCl₄) and olive oil was intraperitoneally administered to BALB/c mice at intervals of twice/week for 4 weeks according to a previously reported method. For a control study, a group of healthy mice was constructed by injecting 100% olive oil instead of the carbon tetrachloride-containing mixture for the same period.

6-2: Treatment of Liver Fibrosis Animal Model with Drug Delivery Carrier and/or ASO Complex

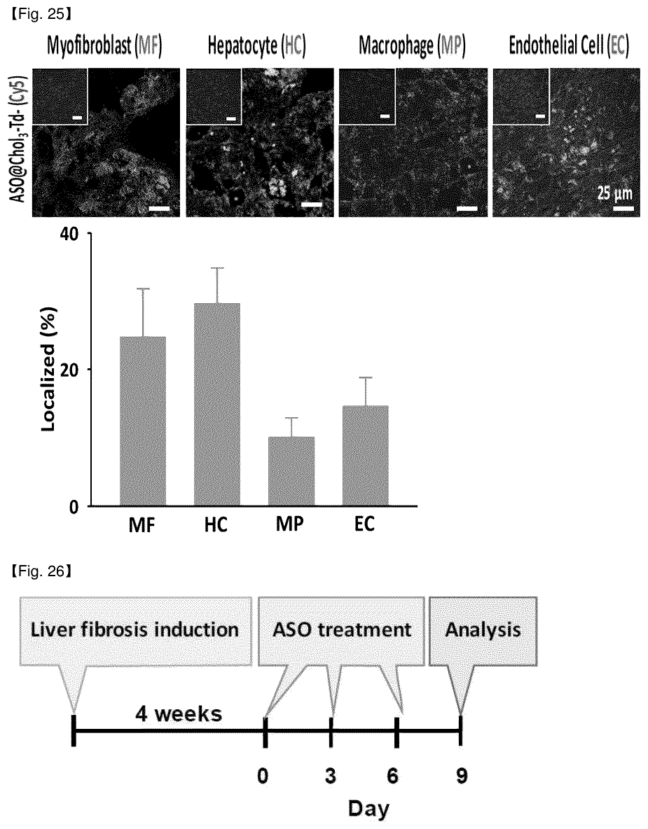

Three days after the last injection of the CCl₄-olive oil mixture, liver fibrosis-induced mice of each group (N=4/group) were intraperitoneally injected with PBS, ASO, ASO@Chol₃-Td (target sequence), or ASO-SC@Chol₃-Td (scrambled sequence) three times at 3-day intervals. Three days after the third injection, blood and major organs were harvested for other analysis. For a control study, PBS-injected healthy mice were subjected to the same sampling procedure. A schematic diagram thereof is illustrated in FIG. 26.

6-3: Imaging Analysis

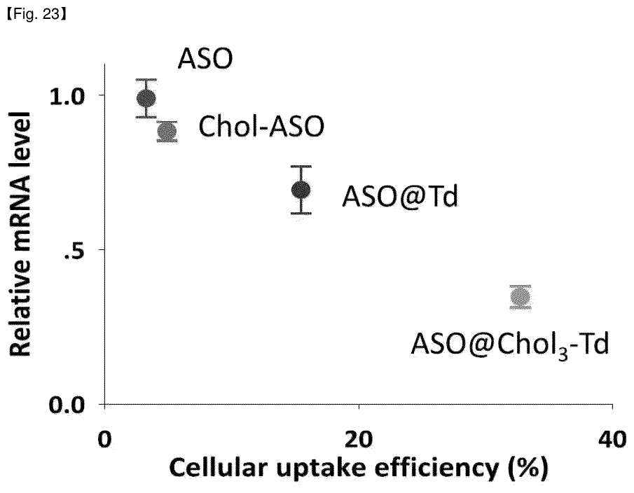

Liver fibrosis animal models were treated with PBS, ASO, or ASO@Chol₃-Td, organs were harvested 2 hours later, ex vivo imaging was performed, and as a result, it was confirmed that ASO@Chol₃-Td is specifically delivered to the liver upon systemic administration, but free ASO is more distributed in the kidney than in the liver (FIG. 24).

This shows that the affinity of ASO@Chol₃-Td to the liver is not impaired by ASO loading and is maintained in the liver fibrosis animal model.

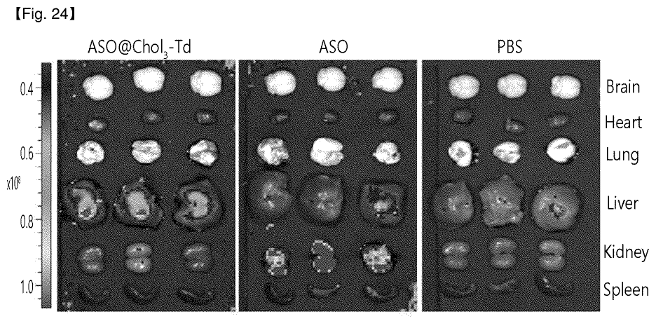

As a result of imaging and quantification of liver sections harvested 2 hours after ASO@Chol₃-Td injection into liver fibrosis mice using a confocal fluorescence microscope, hepatocyte localization of ASO@Chol₃-Td can still be found despite decreases in the number and size of sinusoidal endothelial fenestrae during liver fibrosis (FIG. 25; ASO@Chol₃-Td is labeled with Cy5 (red), liver cell type-specific antibody is labeled with FITC (green), and the yellow region indicates localization of Chol₃-Td).

It was confirmed that Chol₃-Td is also absorbed into myofibroblasts, which are the main cells that produce extracellular matrix (ECM) components causing fibrosis, and this is expected to be because LDLR is highly expressed in myofibroblasts and activated hepatic stellate cells, which are precursors thereof.

The results show that the lipoprotein-associated protein corona of Chol₃-Td is still efficient for targeted delivery even in the fibrotic environment.

6-4: RT-PCR and Western Blotting Analysis

For the treatment of liver fibrosis, ASO@Chol₃-Td or ASO was intraperitoneally injected into disease mouse models every 3 days as in Example 6-2. The therapeutic effect of ASO@Chol₃-Td was examined 3 days after the third injection.

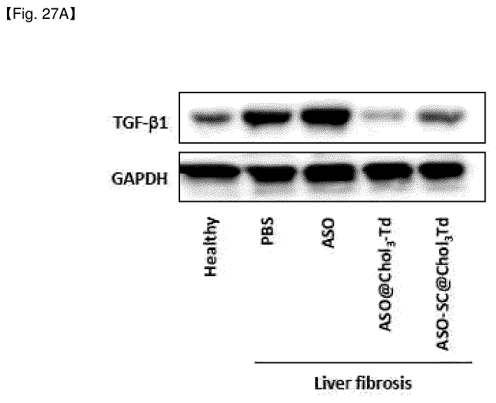

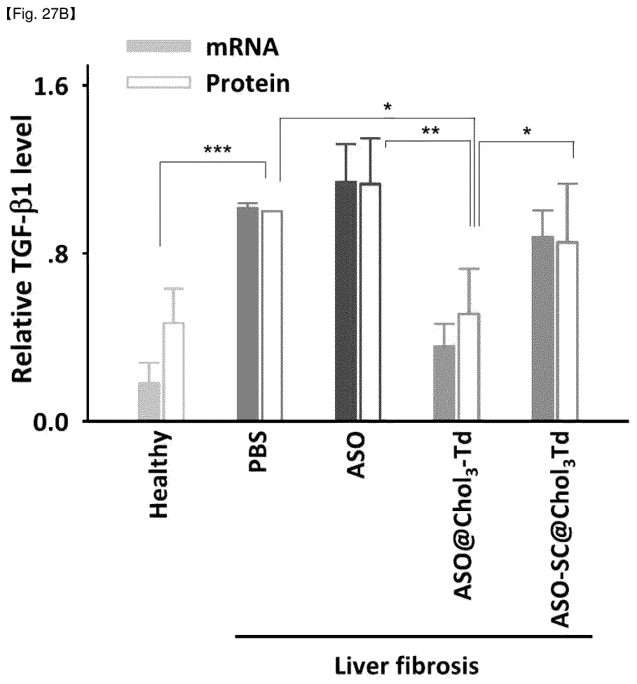

At this time, both the TGF-β1 mRNA and protein levels decreased (FIG. 27).

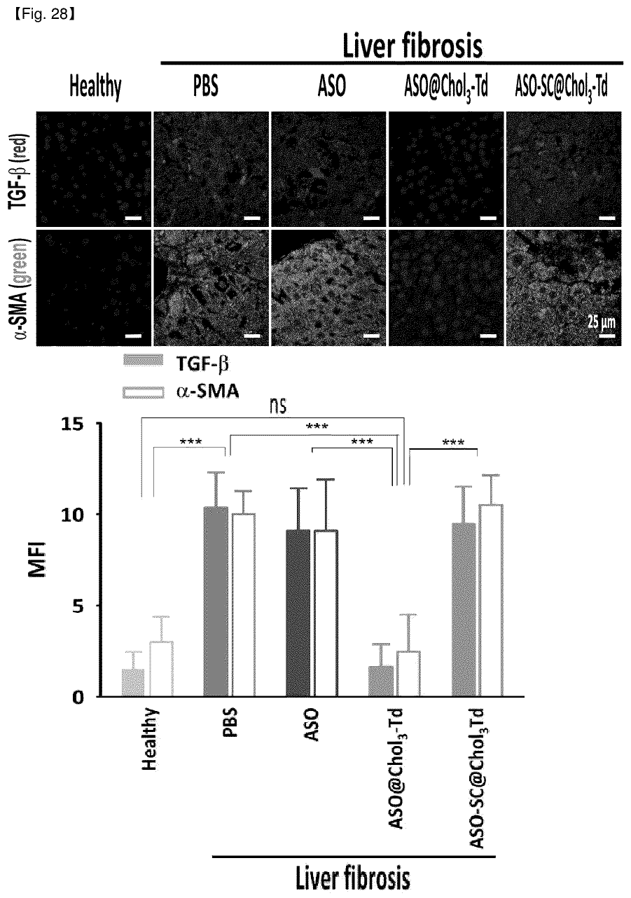

Similarly, when liver TGF-β1 expression was imaged by immunofluorescence microscopy, it was confirmed that the target protein level decreases only after the treatment with ASO@Chol₃-Td (FIG. 28).

Meanwhile, target mRNA expression in the liver was not affected by ASO or ASO-SC@Chol₃-Td, and it was thus confirmed from the in vivo administration of ASO that only Chol₃-Td and the target sequence remarkably greatly affect the delivery ability to the liver (FIGS. 27 and 28).

In addition, uptake of ASO@Chol₃-Td into macrophages and endothelial cells also lowers the TGF-β1 levels and their secretion in these cells.

After chronic liver damage, TGF-β1 secreted from hepatocytes activates hepatic stellate cells to differentiate the hepatic stellate cells into myofibroblasts, and induces differentiation of endothelial cells (differentiation of hepatocytes into myofibroblasts). In other words, when the expression level of TGF-β1 is generally decreased in the liver, the number of α-SMA-expressing myofibroblasts decreases (FIGS. 25 and 28).

### Example 7: Evaluation of Fibrosis Reducing Ability of Complex Containing Nucleic Acid Nanoparticle, Cholesterol, and Pharmacologically Active Ingredient in Liver Fibrosis Animal Model

7-1: Histological Analysis and Immunofluorescence Staining Analysis

In order to measure liver damage, a mouse-derived tissue was immobilized in 10% formaldehyde solution, embedded in paraffin, cut into 4 μm, and stained with hematoxylin and eosin (H & E). In order to measure the level of collagen I, liver sections were stained with Sirius red. The stained tissue slides were prepared at Lab Core incorporation, and measured using an optical microscope (Eclipse Ti-S, Nikon, Tokyo, Japan). For quantitative analysis of collagen fibers stained with Sirius red (red part of image), 20 images (4 mice/group, 5 random parts/mouse) were taken and analyzed by % Area using Image J software. For liver immunofluorescence imaging, deparaffinized sections were boiled in 0.1 M citrate buffer (pH 6) for 20 minutes and incubated with 0.3% (v/v) hydrogen peroxide in methanol for 15 minutes. The sections were blocked with 5% BSA solution at room temperature for 1 hour and incubated with primary antibodies against TGF-β1, α-SMA, F4/80 and CD-31 (1:200, Santa Cruz Biotechnology) at 4° C. overnight. Thereafter, the cells were treated with a fluorescent labeled secondary antibody (1:1000, Cell Signaling Technology) at room temperature for 1 hour. The slides were constructed using a confocal microscope (LSM800) and quantified using Image J software. The MFI values (yellow-orange) of common regions where red represented TGF-β and green represented macrophages or endothelial cells were shown in the graph calculated from the analysis of 12 images (4 mice/group, 3 random parts/mouse).

As a result, in H & E (hematoxylin and eosin) staining-based histological analysis, it was confirmed that acute necrosis and inflammatory cells near the portal vein caused by chronic damage are significantly decreased by ASO@Chol₃-Td.

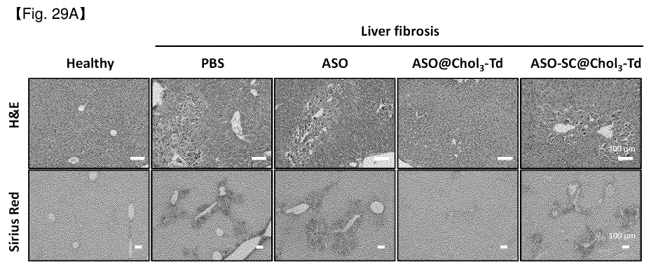

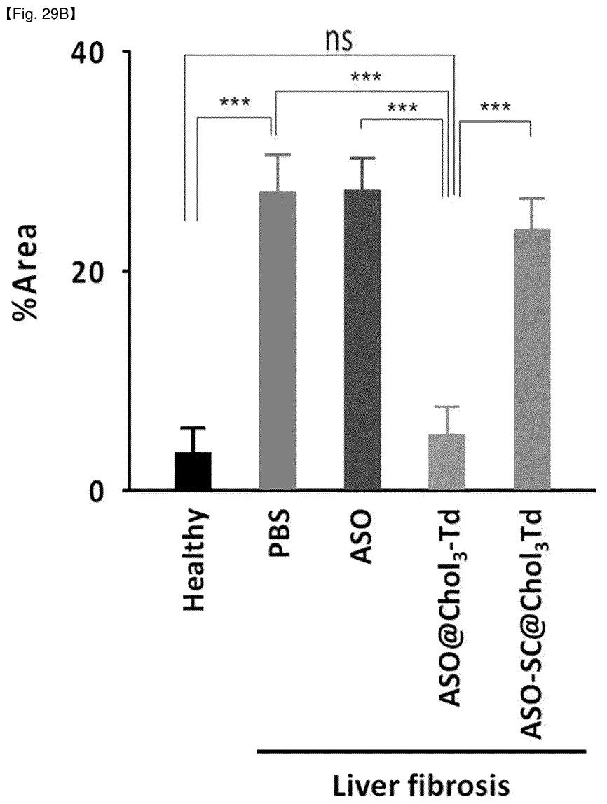

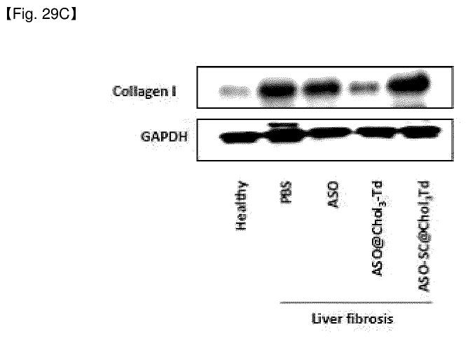

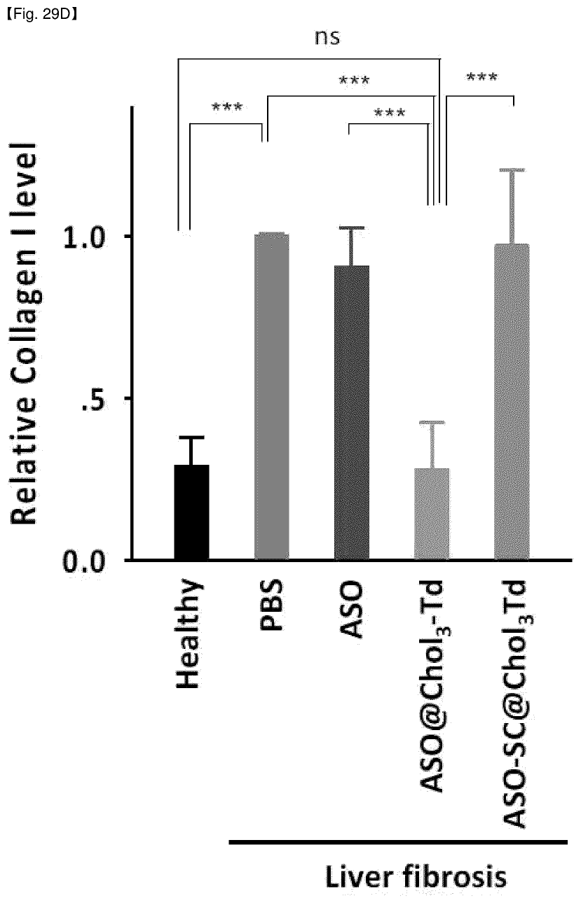

In addition, when measured by Sirius red staining of liver sections and Western blotting of tissue lysates, it was confirmed that the therapeutic effect of ASO@Chol₃-Td on liver fibrosis can be supported by decreases in collagen, proteins of the TGF-β1 signaling pathway, and important components of extracellular matrix of fibrotic liver tissue (FIG. 29).

Meanwhile, ASO and ASO-SC@Chol₃-Td have failed to decrease TGF-β1 expression, liver tissue damage, and collagen levels.

7-2: Serum Analysis

At the end of treatment, blood samples were taken from the mice (4 individuals per group). The samples were coagulated at room temperature for 30 minutes and centrifuged at 4500 rpm and 4° C. for 15 minutes. The amounts of AST and ALT in the supernatant were analyzed using SCL Healthy Inc. (Yongin, Korea).

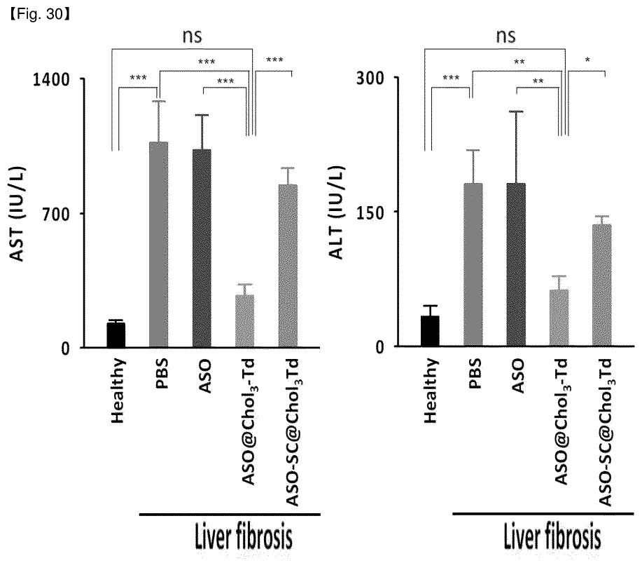

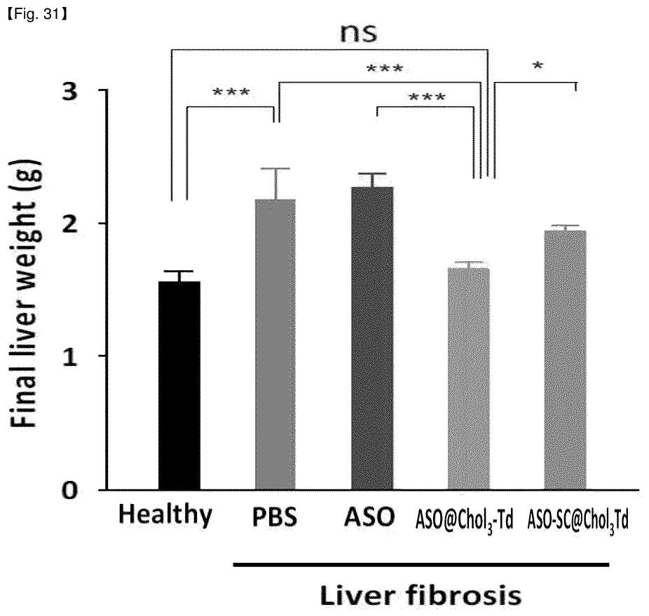

Reduction of liver fibrosis due to the treatment with ASO@Chol₃-Td was examined by a decrease in phenotypic serum markers for liver damage, such as aspartate aminotransferase (AST) and alanine aminotransferase (ALT), and as a result, it was confirmed that the weight of the liver, which has increased abnormally by liver fibrosis, is restored to the level of healthy mice by the therapeutic effect of ASO@Chol₃-Td (FIGS. 30 and 31).

From the results, it was confirmed that the complex of an antisense oligonucleotide for TGF-β1, cholesterol, and a nucleic acid nanoparticle is liver-specific and has a therapeutic effect on liver fibrosis.

### Example 8: Evaluation of Efficacy Compared to Known Drug Delivery Carrier

8-1: Synthesis and Purification of Complex (GalNAc₃-ASO) of GalNAc₃ and ASO

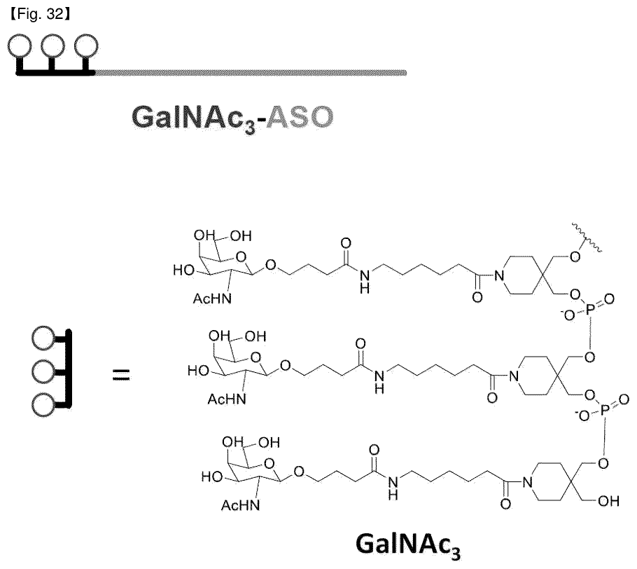

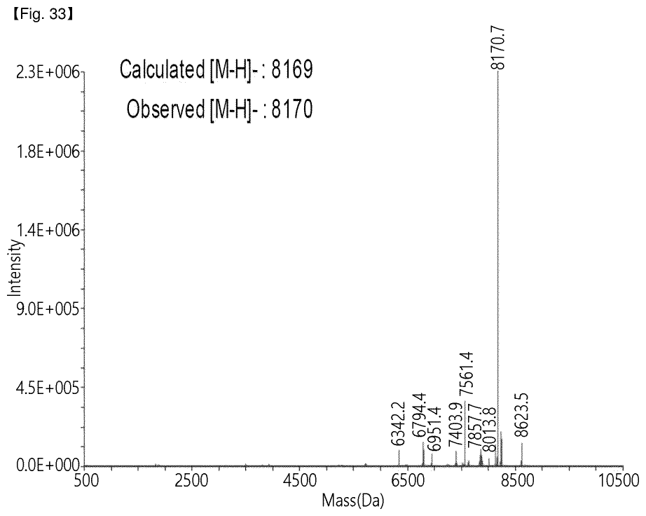

A complex (5′-GalNAc-modified ASO) of 5′-GalNAc and modified ASO was synthesized at 1 pmol scale using standard phosphoramidite chemistry and the Mermaid-4 DNA/RNA synthesizer (Bioautomation, MN, USA). The oligonucleotide was deprotected by cleavage from CPG in 33% ammonia solution at 55° C. for 17 hours, then purified by denaturing PAGE, and ethanol-precipitated. The synthesis of such a complex of GalNAc and modified ASO was confirmed by ESI-MS analysis performed by Novatia Inc. (PA, USA). This was used for the in vitro and in vivo efficiency study compared to a complex (ASO@Chol₃-Td) of ASO, a nucleic acid nanoparticle, and cholesterol. The structures of GalNAc₃ and GalNAc₃-ASO are illustrated in FIG. 32, and the result of electron spray ionization (ESI) mass spectrometry of GalNAc₃-ASO is illustrated in FIG. 33.

8-2: Effect Comparison of GalNAc₃-ASO and ASO@Chol₃-Td

GalNAc₃ (trivalent N-acetylgalactosamine ligand), the latest clinically approved drug delivery platform, and Chol₃-Td were compared to each other.

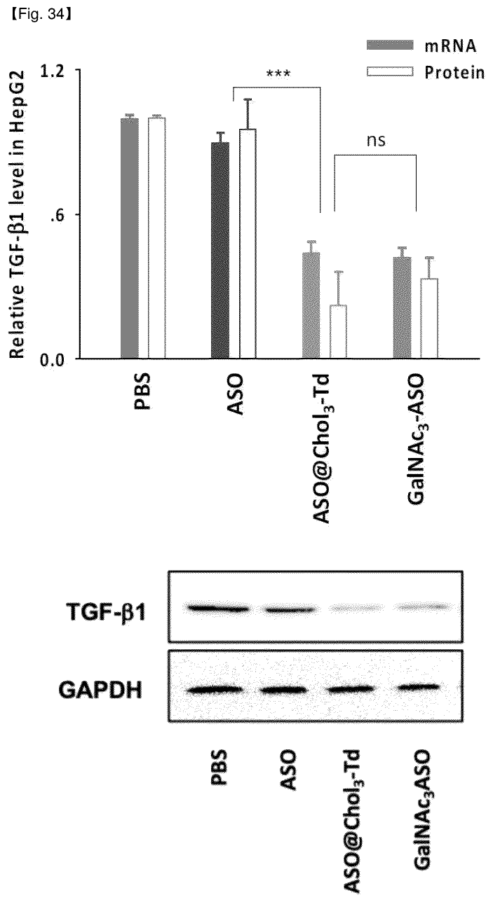

In HepG2 cells, TGF-61 mRNA and protein levels have decreased in both ASO@Chol₃-Td and GalNAc₃-ASO (FIG. 34). From the results, it was confirmed that ASO@Chol₃-Td has remarkable target gene silencing activity as GalNAc₃-ASO does.

Thereafter, in the same manner as in FIG. 26, GalNAc₃-ASO was subcutaneously injected into liver fibrosis mice (the most recommended injection method for GalNAc₃-linked oligonucleotide therapeutic agents).

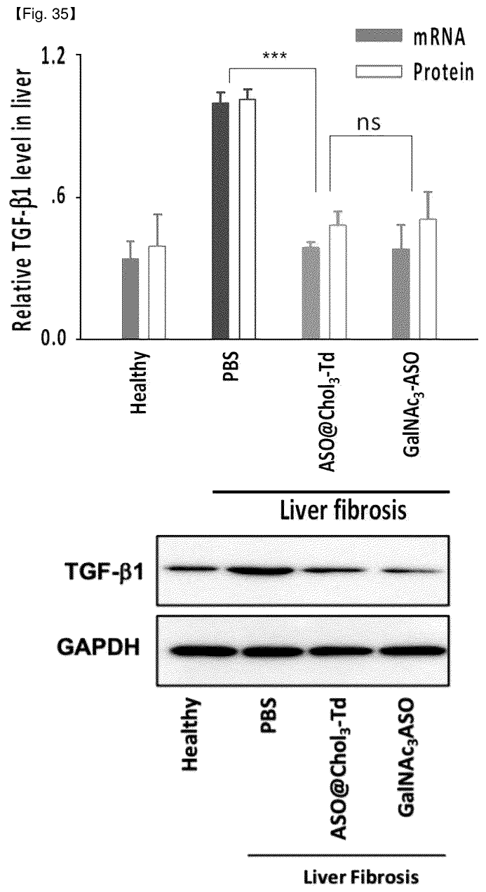

As a result, identical to the cell activity results, the in vivo activity of GalNAc₃-ASO is also similar to that of ASO@Chol₃-Td as both ASO@Chol₃-Td and GalNAc₃-ASO decrease TGF-β1 mRNA and protein levels (FIG. 35).

### Example 9: Comparison of Ability to Reach Liver Tissue Depending on Injection Route (Experiment Related to IV Administration and Oral Administration

9-1. Intravenous Injection

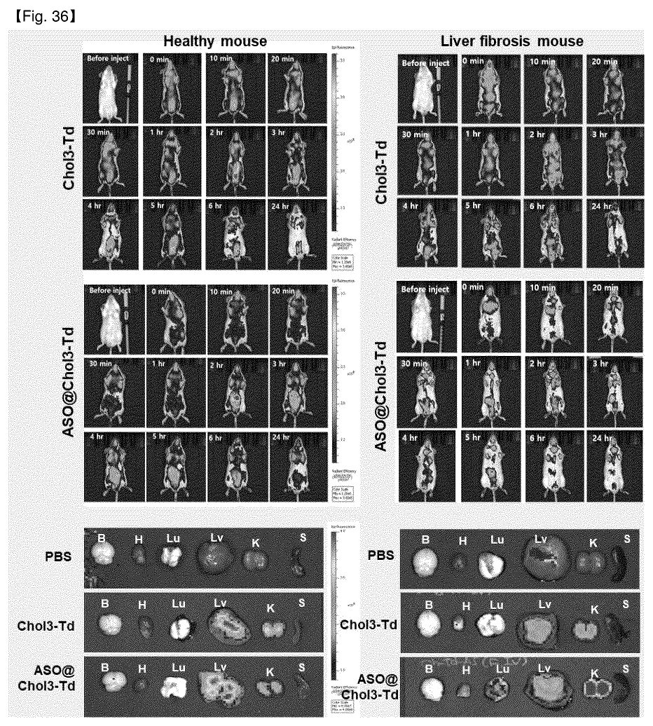

The ability to reach liver tissue was tested according to various injection routes. Chol₃-Td and ASO@Chol₃-Td were injected into healthy mice and liver disease (liver fibrosis) model mice, respectively. After injection through the tail vein of mice, the hourly biological behavior was checked for 24 hours, and at the 2nd hour, the same time as in the intraperitoneal injection, major organs (brain, heart, lung, liver, kidney, and spleen) were harvested and imaged (FIG. 36).

As a result, it was confirmed that there is no change in the properties of Chol₃-Td having liver tissue selectivity after being labeled with ASO in tail vein injection as well.

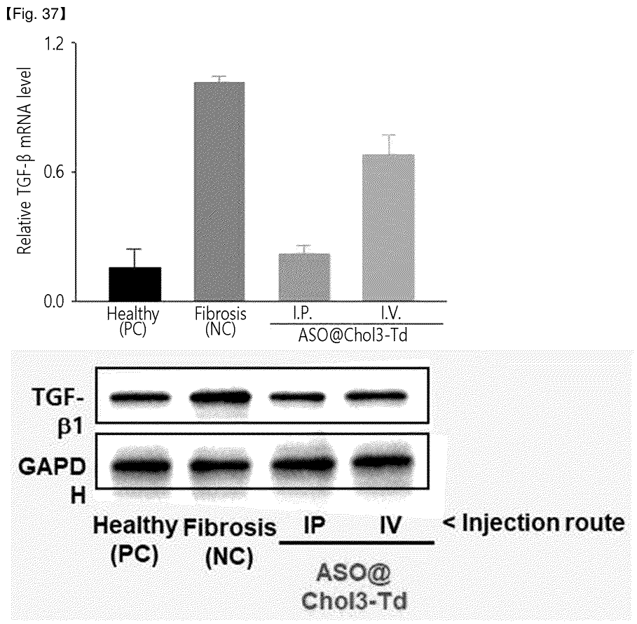

In addition, in the same manner as in FIG. 26, the mRNA of TGF-β1, the target gene, and protein expression levels were examined by RT-PCR and Western blotting after injection into liver fibrosis mice (FIG. 37).

As a result, there is a therapeutic effect as the degree of mRNA expression is decreased by 30% by tail vein injection as well, the target gene is decreased by 70% or more by intraperitoneal injection, and this thus shows that the therapeutic effect by intraperitoneal injection is about 2.5-fold the therapeutic effect by tail vein injection.

9-2. Oral Administration

The ability to reach the liver when Chol₃-Td was orally administered was examined. To this end, the same amount of Chol₃-Td as in the intraperitoneal injection experiment was orally administered using an oral gavage needle, and the in vivo behavior of Chol₃-Td was observed over time. As a control, Chol₃-Td pre-complexed with lipoprotein (HDL) was used. Major organs were harvested and analyzed at the 2nd and 24th hours, and as a result, Chol₃-Td, which was not protected by lipoprotein, also had the ability to reach liver tissue.

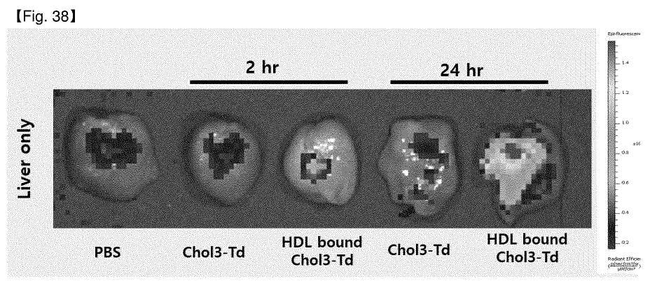

Meanwhile, the liver selectivity was far higher in the samples administered with a protein corona formed in advance (FIG. 38). This shows that the protective effect by lipoprotein helps the structures pass through the acidic stomach, and the undamaged structures undergo protein adsorption through the in vivo circulation, resulting in accumulation in the liver at the 24th hour.

From the results, it was confirmed that the complex of cholesterol and a nucleic acid nanoparticle can be a liver-specific drug delivery carrier and can be used as an effective platform for treatment of liver disease.

From the above description, those skilled in the art to which the present invention pertains will be able to understand that the present invention may be embodied in other specific forms without changing the technical spirit or essential characteristics thereof. Therefore, it should be understood that the embodiments described above are not limitative but are illustrative in all respects. The scope of the present invention is defined by the appended claims rather than by the description preceding them, and all changes or modifications derived from the meaning and scope of the claims and their equivalent concepts should be construed as being included in the scope of the present invention.

1. A liver-specific drug delivery carrier comprising a nucleic acid nanoparticle and cholesterol.

2. The drug delivery carrier according to claim 1, wherein the cholesterol is linked to the nucleic acid nanoparticle.

3. The drug delivery carrier according to claim 1, wherein the drug delivery carrier comprises 1 to 4 cholesterol molecules.

4. The drug delivery carrier according to claim 1, wherein the nucleic acid nanoparticle has a three-dimensional structure.

5. The drug delivery carrier according to claim 1, wherein the nucleic acid nanoparticle is composed of a tetrahedral structure.

6. The drug delivery carrier according to claim 1, wherein the nucleic acid nanoparticle is composed of any one or more oligonucleotides selected from the group consisting of nucleotide sequences of SEQ ID NOs: 1 to 8.

7. The drug delivery carrier according to claim 6, wherein the nucleic acid nanoparticle is composed of four oligonucleotides.

8. The drug delivery carrier according to claim 1, wherein the drug delivery carrier forms a protein layer by being bound to a serum protein.

9. The drug delivery carrier according to claim 1, wherein the drug delivery carrier further comprises a pharmaceutically active ingredient.

10. The drug delivery carrier according to claim 9, wherein the pharmaceutically active ingredient is linked to the nucleic acid nanoparticle.

11. The drug delivery carrier according to claim 9, wherein the pharmaceutically active ingredient includes a nucleic acid.

12. The drug delivery carrier according to claim 9, wherein the pharmaceutically active ingredient is any one or more selected from the group consisting of aptamer, sRNA, miRNA, mRNA, shRNA, CIRSPR/Cas, and antisense oligonucleotide (ASO).

13. A liver-specific complex comprising a nucleic acid nanoparticle and cholesterol.

14. A pharmaceutical composition for prevention or treatment of liver disease, comprising the drug delivery carrier according to claim 1 and a pharmaceutically active ingredient.

15. The pharmaceutical composition according to claim 14, wherein the liver disease is any one or more selected from the group consisting of liver damage, liver fibrosis, liver inflammation, liver cirrhosis, hepatitis, liver decompensation, steatosis, and liver cancer.

16. A method for preventing or treating liver disease, the method comprising administering the pharmaceutical composition according to claim 14 to a subject.
